(19) United States
(12) Patent Application Publication
(10) Pub. No.: US 2025/0367468 A1
(43) Pub. Date: Dec. 4, 2025

(54) NANOPLASMONICS-ENHANCED LASER THERAPY SYSTEMS AND METHODS THEREOF

(71) Applicant: Duke University, Durham, NC (US)

(72) Inventors: Tuan VO-DINH, Durham, NC (US); Peter FECCI, Durham, NC (US); Yang LIU, Durham, NC (US); Pakawat CHONGSATHIDKIET, Durham, NC (US); Ren ODION, Durham, NC (US)

(21) Appl. No.: 18/876,245

(22) PCT Filed: Jun. 12, 2023
(86) PCT No.: PCT/US2023/025025

§ 371 Date: Dec. 18, 2024

Related U.S. Application Data
(60) Provisional application No. 63/355,711, filed on Jun. 27, 2022.

(51) Int. Cl. A61N 5/06; A61B 18/00; A61K 41/00; A61N 5/067

(52) U.S. Cl. CPC A61N 5/062; A61K 41/0052; A61N 5/0625; A61N 5/067; A61B 2018/00321; A61N 2005/0659

(57) ABSTRACT

A method of selectively heating a lesion or tumor using laser therapy treatment includes administering plasmonic metal nanoplatforms to a subject having the lesion or tumor and administering laser therapy treatment to the lesion or tumor. The plasmonic metal nanoplatforms selectively accumulate in the lesion or tumor and the laser therapy is strongly absorbed by the plasmonic metal nanoplatforms that are accumulated in the lesion or tumor. The plasmonic metal nanoplatforms fill the contours of the lesion or tumor thereby enabling laser treatment in a conformal way. The plasmonic metal nanoplatforms selectively accumulate in the lesion or tumor produce efficient photon-to-heat conversion, thus transforming them into heat sources, leading to efficient heat transport, thereby inducing a larger treatment area.

### CROSS-REFERENCE TO RELATED APPLICATIONS

This application claims priority to U.S. Provisional Patent Application No. 63/355,711, filed on Jun. 27, 2022, which is incorporated by reference herein in its entirety.

### BACKGROUND

Light is a non-invasive means for several important medical treatments, including photothermal therapy. Recent technological advances include the minimally invasive use of lasers to thermally ablate lesions or tumors. However, intracranial tumors remain a challenge to the progress of modern oncologic therapies. Few substantial evolutions have occurred in the treatment of either primary or metastatic brain tumors with current mainstays including surgical resection and chemoradiation, with some targeted, systemic, and immunotherapeutic options based on specific tumor histologies.

There are several known methods using light to excite photoactive compounds non-invasively for medical treatment. Light having wavelengths within the so-called “therapeutic window” (700-1300 nm) can be used. The ability of light to penetrate tissues depends on absorption. Within the spectral range known as the therapeutic window (or diagnostic window), most tissues are sufficiently weak absorbers to permit significant penetration of light. This window extends from 600 to 1300 nm, from the orange/red region of the visible spectrum into the NIR. At the short-wavelength end, the window is bound by the absorption of hemoglobin, in both its oxygenated and deoxygenated forms. The absorption of oxygenated hemoglobin increases approximately two orders of magnitude as the wavelength shortens in the region around 600 nm. At shorter wavelengths many more absorbing biomolecules become important, including DNA and the amino acids tryptophan and tyrosine. At the infrared (IR) end of the window, penetration is limited by the absorption properties of water. Within the therapeutic window, scattering is dominant over absorption, and so the propagating light becomes diffuse, although not necessarily entering the diffusion limit.

Laser interstitial thermal therapy (LITT) represents one promising development with its validated use in the treatment of primary and metastatic intracranial tumors, as well as inflammatory post-radiation treatment effect and epilepsy. LITT uses imaging-derived stereotactic guidance to precisely place a catheter within a lesion or tumor for both diagnostic tissue sampling and delivery of thermally ablative energy via a laser probe. Beyond the direct cytotoxic effects of ablation, there is also evidence that such thermal therapy sensitizes tumors to further treatment and triggers a systemic anti-cancer immune response. However, a limitation on its use is due to the non-uniformity of specific heat across the various intracranial tissues, leading to differential conduction of heat within the tumor, surrounding white and gray matter, and regional heat sinks such as blood vessels and cerebrospinal fluid (CSF) spaces. These differences limit the size of candidate lesions or tumors to generally under 3 cm and can lead to an ablated tissue volume that does not accurately conform to the tumor margins.

LITT therapy is safe and effective but cannot treat large and complex tumors. Widespread use of this promising technology suffers from several limitations, with the most prominent being the size of treatable lesions or tumors (roughly 3 cm through a single trajectory) and the lack of specific conformity to tumor margins. Significant pitfalls thus include either incomplete treatment or collateral damage to healthy tissues beyond tumor margins, because of limited light penetration and non-uniform thermal properties in intracranial tissues. Both shortcomings can be addressed with the present disclosure, which combines laser therapy with plasmonic metal nanoplatforms and has the potential to address these challenges.

### SUMMARY

In a first aspect of the present invention, a method of selectively heating a lesion or tumor using laser therapy treatment comprises administering plasmonic metal nanoplatforms to a subject having the lesion or tumor, whereby said plasmonic metal nanoplatforms selectively accumulate in the lesion or tumor, and after selective accumulation of the plasmonic metal nanoplatforms in the lesion or tumor, administering laser therapy treatment to the lesion or tumor.

In a second aspect of the invention, a method of protecting heathy tissue near a lesion or tumor during laser therapy of the lesion or tumor comprises administering plasmonic metal nanoplatforms to a subject having the lesion or tumor, whereby said plasmonic metal nanoplatforms selectively accumulate in the lesion or tumor, and after selective accumulation of the plasmonic metal nanoplatforms in the lesion or tumor, treating the lesion or tumor with laser therapy whereby the laser therapy is strongly absorbed by the plasmonic metal nanoplatforms accumulated in the lesion or tumor thereby reducing destruction of healthy tissue near the lesion or tumor.

In a third aspect of the invention, a method of enhancing absorption of excitation light from laser therapy in a lesion or tumor being treated with said laser therapy comprises administering plasmonic metal nanoplatforms to a subject having the lesion or tumor to be treated with laser therapy, whereby said plasmonic metal nanoplatforms selectively accumulate in the lesion or tumor, and treating the lesion or tumor with laser therapy whereby the excitation light from the laser therapy is strongly absorbed by the plasmonic metal nanoplatforms accumulated in the lesion or tumors.

In a fourth aspect of the invention, a method of accelerating heating rate of a lesion or tumor being treated with laser therapy comprises administering plasmonic metal nanoplatforms to a subject having the lesion or tumor to be treated with laser therapy, wherein the plasmonic metal nanoplatforms selectively accumulate in the lesion or tumor and wherein the plasmonic metal nanoplatforms absorb photons from laser therapy at a higher rate than tissue in the lesion or tumor, and treating the lesion or tumor with laser therapy whereby photons from the laser therapy are absorbed by the plasmonic metal nanoplatforms accumulated in the lesion or tumor thereby accelerating the heating rate of the plasmonic metal nanoplatforms thus accelerating heating of the lesion or tumor in which the plasmonic metal nanoplatforms are accumulated.

In a fifth aspect of the invention, a method of enhancing absorption of excitation light from laser therapy in a lesion or tumor being treated with said laser therapy comprises administering plasmonic metal nanoplatforms to a subject having the lesion or tumor to be treated with laser therapy, whereby said plasmonic metal nanoplatforms selectively accumulate in the lesion or tumor having different and/or irregular shapes by filling the contours of the lesion or tumor, and treating the lesion or tumor in a conformal way with laser therapy whereby the excitation light from the laser therapy is strongly absorbed by the plasmonic metal nanoplatforms accumulated in the lesion or tumor thereby filling contours of the lesion or tumor.

In a sixth aspect of the invention, a method of enhancing absorption of excitation light from laser therapy in a lesion or tumor being treated with said laser therapy comprises administering plasmonic metal nanoplatforms to a subject having the lesion or tumor to be treated with laser therapy, whereby said plasmonic metal nanoplatforms selectively accumulate in the lesion or tumor, and treating the lesion or tumor with laser therapy whereby the excitation light from the laser therapy is absorbed by the plasmonic metal nanoplatforms accumulated in the lesion or tumor thereby inducing photon-to-heat conversion in the plasmonic metal nanoplatforms, thus transforming them into heat sources, leading to further heat propagation in tissue, lesion or tumor and leading to efficient heat transport thereby inducing a larger treatment area. In this way, the GNS enable a doctor or surgeon to use lower laser energy when performing laser treatment or ablation on a tumor because the GNS convert and amplify the laser light into heat thereby enhancing the treatment efficiency.

In a feature of the various aspects, the plasmonic metal nanoplatforms are selected from the group consisting of metal nanostars, metal nanorods, metal nanocaps, metal nanoshells, nanospheres, nanocages, nanotriangles, nanoplates. In another feature of the aspects, the metal of the plasmonic metal nanoplatforms comprises gold, silver, copper or a combination thereof. In a further feature of the aspects, the concentration and selection of metal in the plasmonic metal nanoplatforms is chosen for plasmon tunability. For example, plasmon tunability can comprise adjusting the Ag⁺ concentration of the nanoplatform during synthesis. In this regard, testing has shown that higher concentrations of Ag⁺ progressively red-shift the plasmon band. In another example, gold nanoparticles (e.g., gold nanostars) have a tunable plasmonic absorption band in the near infrared region around 1000 nm, where there is low tissue absorption.

In yet another feature of the various aspects, the plasmonic metal nanoplatforms comprise a bioreceptor. The bioreceptor can comprise DNA probes, antibody probes, enzyme probes, cell receptors, or peptides that are used to help target the lesion or tumor with exquisite specificity. In an additional feature of the aspects, the plasmonic metal nanoplatforms are administered via infusion. In a further feature of the aspects, the laser therapy treatment comprises Laser Interstitial Thermal Therapy (LITT). The LITT treatment can be used in conjunction with Magnetic Resonance Imaging (MRI). In various aspects, the lesion or tumor comprises a tumor. Moreover, the tumor may be an intracranial tumor.

### BRIEF DESCRIPTION OF THE DRAWINGS

The accompanying Figures and Appendix are provided by way of illustration and not by way of limitation. The foregoing aspects and other features of the disclosure are explained in the following description, taken in connection with the accompanying example figures (also “FIG.”) relating to one or more embodiments.

FIG. 1A is a cross-sectional MRI of a patient with a metastatic brain tumor undergoing laser interstitial thermal therapy (LITT).

FIG. 1B includes intra-operative MRI images from 4 patients with metastatic brain tumors who underwent laser interstitial thermal therapy (LITT).

FIGS. 2A-2H provide a non-exclusive schematic representation of various nanostar embodiments. FIG. 2A shows a plasmonics-active metal nanostar. FIG. 2B shows a nanostar labeld with drug and dye molecules. FIG. 2C shows a nanostar with a layer, embedded with a label and/or a drug. FIG. 2D shows a nanostar with a layer, embedded with a label and/or a drug and a protective layer. FIG. 2E shows a nanostar with a paramagnetic spherical nucleus. FIG. 2F shows a nanostar with an elongated paramagnetic nucleus. FIG. 2G shows a void space nanostar. FIG. 2H shows a nanostar with an empty or dielectric core.

FIGS. 3A-3H provide a non-exclusive schematic representation of various nanostars with bioreceptors. FIG. 3A shows a plasmonics-active metal nanostar with a bioreceptor. FIG. 3B shows a nanostar labeled with optical dye and/or drug molecules with bioreceptor. FIG. 3C shows nanostars with layer (embedded with label and/or drug) with bioreceptor. FIG. 3D shows a nanostar with layer (embedded with label and/or drug) and protective overlayer with bioreceptor. FIG. 3E shows a nanostar with paramagnetic spherical nucleus with bioreceptor. FIG. 3F shows a nanostar with elongated paramagnetic nucleus with bioreceptor. FIG. 3G shows a void-space nanostars with bioreceptor. FIG. 3H shows a nanostar with empty or dielectric core with bioreceptor.

FIG. 4 is a schematic illustration of the synthesis process of nanostars having anisotropically grown gold branches being produced.

FIG. 5 includes a series of TEM images of nanostars formed under different silver ion concentrations.

FIG. 6A shows absorbance spectra (unnormalized) of the star solution (˜2 nM) in citrate buffer versus wavelength for the different Ag⁺ concentrations.

FIG. 6B shows FEM generated absorption spectra of nanostars embedded in water versus wavelength for the different Ag⁺ concentrations.

FIG. 6 INSET is a photograph of corresponding star solutions showing their coloring.

FIG. 7A shows a Monte Carlo simulation of absorbed photon energy in gray matter from a point source laser.

FIG. 7B shows GNSs contained in tumors can both expand coverage and protect surrounding normal tissue structures in the brain phantom.

FIG. 8A is a normalized energy absorption map around the isotropic point source within the layer mimicking gray matter brain tissue.

FIG. 8B is an energy absorption map of tissue having a tumor near the excitation laser point source.

FIG. 9A is a chart showing wavelengths in the optical window of tissue and absorption spectra of biological components.

FIG. 9B shows the transmission emission microscopy (TEM) image of GNS engineered to have absorption around 1064 nm within the tissue window.

FIG. 9C shows the absorption spectrum of GNS engineered to have absorption around 1064 nm within the tissue window.

FIG. 10A is a gross specimen of mouse brain with brain tumor shown in black color due to the uptake of light absorbing GNS nanoparticles.

FIG. 10B is a PET/CT scan of brain-tumor bearing mouse 48 hours after 124I labeled GNS IV injection.

FIG. 11 is a set of images showing that GNS nanoprobes selectively accumulate in the brain tumor after IV injection.

FIG. 12A shows a diagram of the phantom tumor models.

FIG. 12B shows representative images from thermal monitoring after 12 minutes of heating.

FIG. 12C is a graph of temperatures measured 2 cm from the laser probe tip in the gold nanostar infused model (GNS phantom) and control (Control phantom) during the administration of laser interstitial thermal therapy (LITT).

FIG. 12D is a table showing extrapolated and interpolated heating times and temperatures within the tumor phantom (2 cm from the probe tip) using the simple linear regressions calculated from the data in FIG. 12C.

FIG. 12E includes representative images from the M-Vision MRI-thermometry software for the cube and star shaped internal phantoms. The top image is for the cube shaped internal phantom and the bottom image is for the star shaped internal phantom. The adjusted contouring of the heated region provides evidence that the distribution of heat follows the distribution of GNS within the tumor model. The yellow line represents tissue exposed to the equivalent of 43° C. for at least 2 minutes.

FIG. 13A is a schematic image illustrating the phantom prepared for this experiment.

FIG. 13B is a graph of temperatures measured in the split phantom model half infused with gold nanostars (GNS).

FIG. 13C is a table showing extrapolated and interpolated heating times and temperatures of surrounding tissue for various tumor border temperatures using the simple linear regressions calculated from the data in FIG. 6B.

FIG. 14A is a coronal PET/CT scan of brain for tumor-bearing (TB-A, TB-B, TB-C, TB-D) and non-tumor-bearing (NTB) mice at 10 minutes, 24 hours, and 72 hours after intravenous injection of 124I-GNS nanoprobes.

FIG. 14B is a chart showing % injection dose per gram at 10 minutes, 24 hours, and 72 hours after GNS administration.

FIG. 14C provides two images: an autoradiography (left) and H&E histopathology imaging (right) of brain tumor tissue sections from a TB mouse administered intravenous GNS.

FIGS. 15A and 15B are schematic illustrations of the NPE-LITT modality for theranostics of a tumor using gold nanostars.

### DETAILED DESCRIPTION

To promote an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses all possible combinations of one or more of the associated listed items, as well as the lack of combinations when interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated should be considered as expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to affect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient). In some embodiments, the subject comprises a human who is undergoing a procedure using a system or method as prescribed herein.

Unless otherwise defined, all technical terms used herein have the same meaning as commonly understood by one of ordinary skill in the art to which this disclosure belongs.

Described herein is a method of using laser therapy to selectively heat a lesion or tumor being treated with laser therapy. The method comprises administering plasmonic metal nanoplatforms to a subject having the lesion or tumor, whereby said plasmonic metal nanoplatforms selectively accumulate in the lesion or tumor, and then after the plasmonic metal nanoplatforms have accumulated in the lesion or tumor, administering laser therapy to the lesion or tumor. In embodiments, the described method protects healthy tissue near and/or surrounding the lesion or tumor during laser therapy of the lesion or tumor. Moreover, in embodiments, the described method enhances absorption of excitation light from laser therapy in the lesion or tumor being treated with the laser therapy and accelerates the heating rate of the lesion or tumor being treated with laser therapy.

An embodiment of the method includes Nanoplasmonics-Enhanced Laser Interstitial Thermal Therapy (NPE-LITT). NPE-LITT combines LITT with gold nanoparticles that act as “lightning rods” to attract laser light. Advantageously, NPE-LITT can expand the application of laser treatment by delivering heat more selectively and efficiently than is possible in normal tissue. Plasmonic gold nanoparticles selectively accumulate within tumors (e.g., intracranial tumors) due to the EPR effect and can both expand the area effectively treated with laser therapy and protect surrounding heathy tissue. Briefly, the enhanced permeability and retention effect (EPR effect) describes a universal pathophysiological phenomenon and mechanism in which macromolecular compounds can progressively accumulate in a tumor vascularized area and thus achieve targeting delivery and retention of anticancer compounds into solid tumor tissue. The EPR effect has been well observed and documented in solid tumors of rodents, rabbits, canines, and human patients. The synergism of LITT and plasmonics-active gold nanostars (GNS) presents a solution for the next generation treatment of primary and metastatic brain tumors.

Laser-Induced Thermal Therapy (LITT) is a minimally invasive procedure using lasers in the treatment of various intracranial pathologies. The integration of LITT with imaging methods, such as magnetic resonance imaging (MRI), enables surgeons to operate on lesions or tumors located in deep parts of the brain with accurate estimates of thermal damage. LITT has been useful for cases in which tumors are in difficult to access locations. Lasers are a form of nonionizing radiation that produce a coherent and collimated beam of light energy. The effectiveness of a laser on tissue can be determined by two principles: absorption and scatter. Absorption occurs when the laser energy is converted to heat after its photons hit molecules in the target tissue called chromophores. The energy transfer to chromophores results in the release of heat, allowing photothermal heating to take place, which directly damages adjacent cells and structures. Scatter takes place when the trajectory of the photon is deviated by its interaction with particles in the tissue, resulting in an increased spatial distribution of light. A wavelength is chosen in which photon scatter and absorption optimize tissue heating and penetration of light. Several properties of tissue, such as perfusion, conductivity, specific heat, and density, can also influence how laser light affects tissue ablation. In LITT, laser light is transmitted from a generator to the patient's tissue using optical fibers. The optical fibers reach from the laser source located outside of the MRI suite to the patient. Laser light is introduced into the patient through a diffusing tip that is approximately 1 cm in length. Diffusing tips radiate light in a cylindrical to ellipsoid distribution along the axis of the tip. The NeuroBlate system, which will be described in more detail below, uses a 12 W, 1064-nm Nd:YAG laser. The optical fibers are housed inside a catheter sheath to ensure proper cooling of the fiber and clean energy dispersal. Cooling mechanisms vary between LITT systems, however, the NeuroBlate system uses a sapphire capsule with an internal cooling mechanism using CO₂ gas. NeuroBlate catheters come in both 2.2 mm and 3.3 mm diameters.

Thermal effects on tissue from laser treatment include DNA and protein denaturation, ultimately leading to cell death. Up to a temperature of 40° C., the cell can maintain homeostasis; however, temperatures ranging from 46° C. to 60° C. induce irreversible damage to cellular structures. At temperatures greater than 60° C., cells undergo instantaneous protein coagulation, resulting in coagulation necrosis.

This disclosure describes embodiments using gold nanostars combined with LITT to treat brain tumor as the model system. However, the invention is not limited to the brain tumor model system. Other intracranial model systems could include, but not be limited to epileptic foci, tubers, cavernous malformations, arteriovenous malformations, and abscesses. Moreover, lesions and tumors in other locations in the body may also be treated using the methods and systems described herein. Furthermore, the systems and methods described herein could be used with other plasmonics-active nanosystems and could be applied to other cancers and diseases.

Hyperthermia (HT) is a treatment method where heat is applied to a lesion or tumor. A lesion or tumor is generally understood to mean a region in an organ or tissue that has suffered damage through injury or disease. A lesion or tumor can include a tumor or injured or diseased organ but is not limited thereto. While the term tumor is often used in this disclosure, the skilled person will understand that the treatment and methodology described herein may be applicable to other types of lesions or tumors. As a principle, hyperthermia aims to increase tumor temperature above physiologic body temperature (˜36° C.) with the goal of directly inducing cellular damage to abrogate growth, as well as promote local and systemic antitumor immune effects. When a tumor is heated, several important vascular physiological effects occur, including vasodilation, which increases blood flow to the tumor and adjacent tissues. In the case of brain tumors, even a mild increase of local temperature dramatically enhances Blood-Brain-Barrier (BBB) permeability allowing the passage of large therapeutic molecules, including large monoclonal antibodies such as the ones used in immune-blockage inhibition immunotherapy. Increased local vascular and BBB permeability not only improves drug delivery but the ability of immune cells to migrate into the heated tissue; this translates to an improved systemic antitumor immune response.

Current treatment options for brain tumors are limited and include surgical resection, whole-brain radiation therapy (WBRT), stereotactic radiosurgery (SRS), chemotherapy, and targeted therapy. Moreover, there have been no major treatment advances in over two decades. As described above, LITT is an emerging standard-of-care treatment for patients having intracranial tumors. LITT is minimally invasive technique and uses a stereotactically-guided laser to apply heat to tumors, resulting in cell death. LITT has proven capable of temporarily opening the BBB, suggesting it may improve access and efficacy for other modalities, including immunotherapies.

However, due to non-uniformity of specific heat across different intracranial tissues, the current technology cannot deliver a sufficiently large ablation volume or one that specifically conforms to tumor margins. As the skilled person would appreciate, the tumor, surrounding white and gray matter, and regional heat sinks, such as blood vessels and ventricles, all conduct heat differently leading to inhomogeneity, and thus treatment difficulty, when performing laser heat treatment. The non-conformational treatment can lead to incomplete penetration of thermal ablation across the tumor volume and/or collateral damage to healthy tissues beyond the tumor margins.

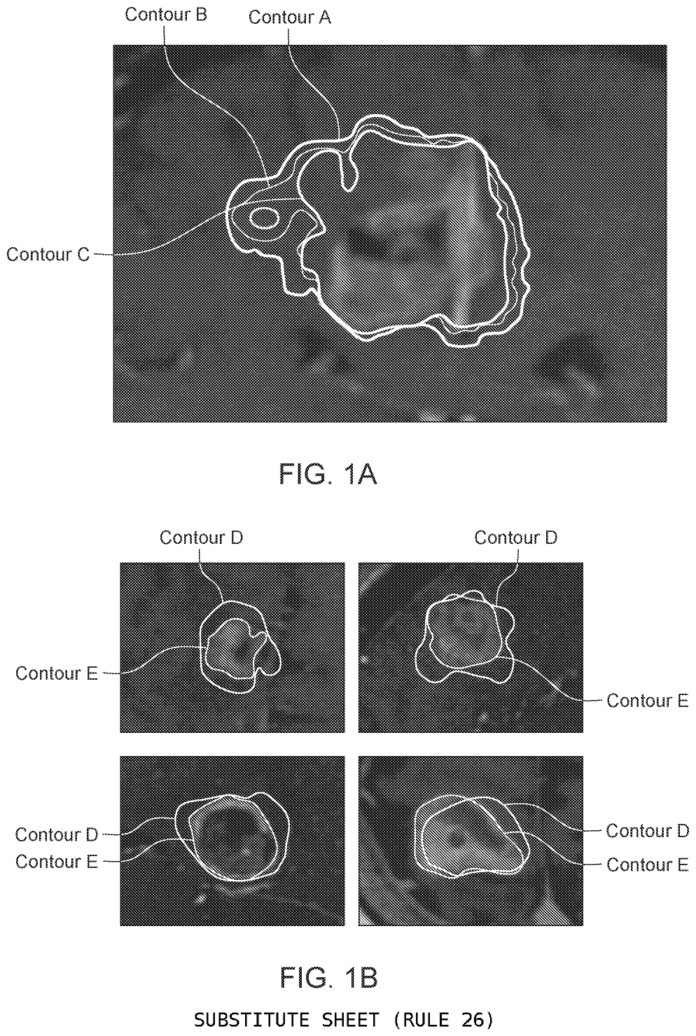

LITT technology development enabled the ability to both accurately target lesions or tumors through a minimally invasive access point and, in real-time, monitor exact changes in temperature of the target and surrounding brain during administration of photothermal energy. An exemplary LITT system is the NeuroBlate System, which can be used in conjunction with M-Vision software. FIGS. 1A-1B provide images of the NeuroBlate System with M-Vision software in use. FIG. 1A is a cross-sectional MRI of a patient with a metastatic brain tumor undergoing laser interstitial thermal therapy (LITT).

Thermal damage threshold (TDT) lines are depicted in the images. The contour line A indicates tissue heated the equivalent of 43° C. for at least 2 minutes (no permanent damage), the contour line B indicates tissue heated the equivalent of 43° C. for 10 minutes (severely damaged), and the contour line C indicates tissue heated the equivalent of 43° C. for 60 min (coagulative necrosis). FIG. 1B includes intra-operative MRI images from 4 patients with metastatic brain tumors who underwent laser interstitial thermal therapy (LITT). The contour line E indicates the borders of the contrast-enhancing tumor volume. The contour line D indicates the blue thermal damage threshold (TDT) boundary, identifying tissue heated to the equivalent of 43° C. for 10 minutes and considered ‘severely damaged’.

As shown in the images in FIGS. 1A and 1B, intra-operatively, the extent of thermal ablation can be displayed by the NeuroBlate System M-Vision software as thermal-damage-threshold (TDT) lines. As described above, the variable heat conduction across tissue structures within and around the tumor complicates uniform coverage of the target lesion or tumor with exclusion of surrounding tissue. The non-conforming treatment illustrated in FIGS. 1A and 1B leads to incomplete penetration of the targeted lesions or tumors and/or collateral damage to healthy tissues beyond its margins. Identifying strategies to increase the specificity of LITT and protect surrounding healthy structures are important next steps in the development of this treatment paradigm.

Plasmonics-active metallic nanostructures have been researched for a wide variety of applications. Plasmonics refers to the study of enhanced electromagnetic properties of metallic nanostructures. The term is derived from plasmons, the quanta associated with longitudinal waves propagating in matter through the collective motion of large numbers of electrons. Molecules on or near metal nanostructures experience enhanced fields relative to that of the incident radiation. When a metallic nanostructured surface is irradiated by an incident electromagnetic field (e.g., a laser beam), conduction electrons are displaced into frequency oscillations equal to those of the incident light. These oscillating electrons, called “surface plasmons,” produce a secondary electric field, which adds to the incident field.

The origin of plasmon resonances of metallic nanoparticles are collective oscillations of the conduction band electrons in the nanoparticles, which are called Localized Surface Plasmons (LSPs). LSPs can be excited when light is incident on metallic nanoparticles having a size much smaller than the wavelength of the incident light. At a suitable wavelength, resonant dipolar and multipolar modes can be excited in the nanoparticles, which lead to a significant enhancement in absorbed and scattered light and enhancement of electromagnetic fields inside and near the particles. Hence, the LSPs can be detected as resonance peaks in the absorption or scattering spectra of the metallic nanoparticles. This condition yields intense localized fields which can interact with molecules in contact with or near the metal surface. In an effect analogous to a “lightning rod” effect, secondary fields can become concentrated at high curvature points on the nanostructured metal surface. Nanoparticles of noble metals such as gold and silver resonantly scatter and absorb light in the visible and near-infrared spectral region upon the excitation of their plasmon oscillations and are therefore materials of choice for plasmon related devices.

Surface plasmons have been associated with important practical applications in surface plasmon resonance (SPR), surface-enhanced Raman scattering (SERS) and surface-enhanced luminescence, also referred to as metal-enhanced luminescence. A wide variety of plasmonics-active SERS platforms have been developed for chemical sensing and for bioanalysis and biosensing. Exemplary platforms include microplates, waveguides or optical fibers having silver-coated dielectric nanoparticles or isolated dielectric nanospheres coated with a silver nanolayer producing nanocaps (i.e., half nanoshells), nanorods and nanostars. The plasmonics substrate platforms have led to a wide variety of analytical applications including sensitive detection of a variety of chemicals of environmental, biological, and medical significance, including polycyclic aromatic compounds, organophosphorus compounds, and compounds of biological interest such as DNA-adduct biomarkers.

The SERS effect can enhance the efficiency of light emitted (Raman or luminescence) from molecules adsorbed or near a metal nanostructures' Raman scatter. The intensity of the normally weak Raman scattering process is increased by factors as large as 10¹³ or 10¹⁵ for compounds adsorbed onto “hot spots” on a plasmonics-active substrate, allowing for single-molecule detection. As a result of electromagnetic field enhancements produced near nanostructured metal surfaces, nanoparticles can be used as fluorescence and Raman nanoprobes. The size of nanoparticles and nanoshells can be tuned to the excitation wavelength.

The origin of the 10⁶- to 10¹⁵-fold Raman enhancement primarily arises from two mechanisms: a) an electromagnetic “lightning rod” effect occurring near metal surface structures associated with large local fields caused by electromagnetic resonances, often referred to as “surface plasmons”; and b) a chemical effect associated with direct energy transfer between the molecule and the metal surface.

When a nanostructured metallic surface is irradiated by an electromagnetic field (e.g., a laser beam), electrons within the conduction band begin to oscillate at a frequency equal to that of the incident light. These oscillating electrons, called “surface plasmons,” produce a secondary electric field that adds to the incident field. If these oscillating electrons are spatially confined, as is the case for isolated metallic nanospheres or roughened metallic surfaces (nanostructures), there is a characteristic frequency (the plasmon frequency) at which there is a resonant response of the collective oscillations to the incident field. This condition yields intense localized field enhancements that can interact with molecules on or near the metal surface. The excitation light from the laser therapy can be absorbed by the plasmonic metal thus inducing photon-to-heat conversion in the plasmonic metal nanoplatforms and transforming the nanoplatforms into heat sources. Secondary fields are typically most concentrated at points of high curvature on the roughened metal surface.

Combining plasmonics-active gold nanoparticles (e.g., nanostars (GNS)) with LITT can address the shortcomings of using LITT alone. Using LITT in conjunction with GNS enables selective heating of regions where GNS are located while keeping surrounding tissues at significantly lower temperatures, which is a noteworthy advantage over conventional thermal therapies. In embodiments, the multiple sharp branches of GNS are plasmonics-active (i.e., exhibiting enhanced electromagnetic properties), acting like “lightning rods” to convert and amplify laser light into heat thus transforming the GNS into a heat source. By selectively accumulating within a tumor and amplifying heat delivery of the laser across tiny distances, the GNS offers the ability to extend and “shape” the laser heat field in a manner that accurately conforms to tumor margins. For example, this may be particularly relevant and helpful in situations where the tumor shape is non-uniform. The GNS accumulate in the shape of the non-uniform shaped tumor. When the laser is directed at the tumor, the GNS, in the shape of the tumor, converts the laser light to heat providing a heat source to all areas of the tumor rather than to pointed areas conventionally provided by a single laser beam. In this way, the GNS enable a doctor or surgeon to drill fewer holes into a patient's skull when performing laser treatment or ablation on a tumor because the GNS convert and amplify the laser light into heat thereby spreading and amplifying the area of treatment using fewer laser treatments. As used herein, the term “selectively accumulates”, “selectively accumulating”, or “selective accumulation” means that a relatively large proportion or percentage of the total plasmonics-active nanoparticles collect or gather in the region of the lesion or tumor (e.g., tumor). Selectively accumulates does not mean that all plasmonics-active nanoparticles collect or gather in the region of the lesion or tumor, but rather a suitable amount of the total plasmonics-active nanoparticles to act as a lightning rod to convert and amplify laser light into heat. The skilled person will understand that with the EPR effect, “selectively accumulates” means that there are more nanoparticles in the lesions or tumors than in surrounding healthy tissue. For example, about 5% of the nanoparticles may collect or gather in the lesion or tumor, while the remaining 95% of the nanoparticles are dispersed throughout the body. Because the size of the tumor is very small (e.g., 1 cm³) in comparison to the size of the human body (e.g., ˜62,000 cm³), the local concentration of nanoparticles in the tumor is significantly higher than the concentration of nanoparticles in surrounding healthy tissue and serves to convert and amplify laser light into heat in the tumor, thereby efficiently treating through ablation the tumor and not the surrounding healthy tissue.

By concurrently optimizing heat delivery and bolstering specificity, GNS and LITT offer a synergy for the treatment of intracranial tumors—the opportunity for both safer and more effective treatment.

Furthermore, gold is highly biocompatible, and gold nanoparticles (due to a combination of EPR effect and diminished lymphatic drainage) accumulate preferentially within tumors following intravenous injection. Rapid and precise hyperthermia can be achieved throughout a tumor, without harming tissue beyond tumor margins.

Several factors should be taken into account when considering GNS-mediated LITT treatment: (1) the optical properties of the tissue, (2) the laser excitation wavelength, (3) the absorption efficiency of the GNS platform, and (4) the photon-to-heat conversion of GNS are important factors in thermal therapies that utilize laser irradiation, such as the LITT modality. Applications that use lasers having wavelengths below infrared must contend with limited penetration depth along with off-target absorption and heating. Tissues such as the skin and blood vessels will absorb much of the laser energy before reaching a tumor tissue target for example. Different strategies should be employed to circumvent the limited penetration to deliver enough energy to the tumor site to induce ablation or hyperthermia.

Laser delivery by optical fiber is the most common strategy in which the fiber head is invasively placed near the target area to deliver the laser light directly. Another option is to use optical sources of specific wavelengths of light that are the tissue “optical window”, a narrow wavelength band between 700-1100 nm where there is little tissue absorption. The use of the 1064-nm laser in this study is suitable to excite within the optical window, where tissue components absorb the least and photons can travel deeper in tissue.

GNSs have a tunable plasmonic absorption band in the near infrared region around 1000 nm, where there is low tissue absorption, and therefore they are suitable for LITT-based photothermal treatment. Gold nanostars have a very high photon to heat conversion. Paired with their ability to target tumors via the Enhanced-Permeation and Retention (EPR) effect, the nanoplatform can be used to greatly enhance photothermal therapy.

In the Examples, the optical response of tissue is studied to analyze the resulting heating after laser irradiation via an optical fiber onto a tissue phantom. The spatio-temporal evolution of the aggregate photons' energy in a layer can be modelled using a second order differential equation shown in equations (1) and (2) below.

$$\frac{1}{c} \frac{\partial φ ( r , t )}{δ t} + {μ}_{a} φ ( r , t ) - \frac{1}{3 ( {μ}_{a} + {μ}_{{s}^{′}} )} \frac{{\partial }^{2} φ ( r , t )}{\partial r \partial t} = S ( r , t ) \qquad ( 1 )$$

$$\partial = \sqrt{\frac{1}{3 {μ}_{a ( {μ}_{a} + {μ}_{{s}^{′}} )}}} \qquad ( 2 )$$

In short, the diffusion equation models the concentration of photon energy in a volume as captured by the term φ (it is also known as the fluence rate). This equation describes the position and movement of the photon concentration through time and is dictated mainly by three terms: the absorption coefficient μₐ, the scattering coefficient μₛ, and the light source S. These properties are either intrinsic to the material or dependent on the light source geometry. The second equation is known as the penetration depth and is roughly the inverse of the sum of the absorption and scattering coefficient of the material. It is the depth at which the magnitude of the energy decays to 1/e of its value. One can see that the penetration of a laser is thus inversely proportional to the optical absorption and scattering of the material. A steady-state solution of this diffusion equation can also be simplified to the well-known Beer-Lambert Law with some unit conversion with the optical properties. From there, the optical properties can be measured experimentally using absorption spectroscopy.

According to the Monte Carlo Photon Transport Scheme, the diffusion equation is a simple model and relies on the assumption that the scattering of the material is much greater than the absorption (the material must be turbid). Additionally, the equation breaks down for distances below the mean free path length between interactions lₜ<1/(μₐ+μ_{s′}). The Monte Carlo Modeling of Photon Transport is a numerical method that uses utilizes a stochastic model to estimate ensemble-averaged quantities. In this context, the ensemble of simulated randomly scattered and absorbed photons is simply the photon energy concentration in space and time (the same as the diffusion equation from earlier). The algorithm consists of randomly sampling variables from probability density functions. These include the photon step size, scattering angle, absorption, etc. A specified number of photons are individually launched and tracked, each depositing energy in different voxel coordinates. The final output consists of a photon fluence map that can be converted to an absorption map.

Results of Monte Carlo Simulations of Laser Irradiation of Gold Nanostars in Optically Scattering Tissue. A key goal is modelling the interaction light with tissue once there is a volume of highly absorbing gold nanostars embedded within. This can show the effective use of gold nanostars as a localized heat zone for more specific thermal therapy. The model depicts a homogenous layer of a specified optical property at 1064-nm excitation. Gray matter of brain tissue is chosen as the model tissue as there is a clinical need for more effective treatment of glioblastoma that minimizes off target heating and damage. The absorption and scattering coefficients are 0.56 and 56.8 cm⁻¹ respectively with an anisotropy value g of 0.9.

The optical properties of the gold nanostar were experimentally determined using absorption spectroscopy. Since nanoparticles including GNSs have the tendency to accumulate preferentially in tumors due to the EPR effect, we use the tumor model as an area that contains concentration of GNS. Our studies using mouse model have determined that the GNS concentration in tumors is 20 μg/g GNS, i.e., 0.1 nM using inductively coupled plasma mass spectrometry (ICP-MS) analysis. To match the typical amount of gold nanostar found accumulated in tumors, the concentration of the GNS particles was set to 0.1 nM, which corresponds to an absorption coefficient of about 2 cm⁻¹. The particle was also assumed to have little scattering, so the scattering coefficient was set to 1 cm⁻¹. A collimated beam with a radius of 1 cm and an isotropic point source placed 3 cm within the volume were chosen as sources. These correspond to typical laser irradiation configurations with a standoff beam and a fiber delivered source. A spherical volume containing only the gold nanostar was embedded within the tissue near the source. Each simulation was set to run for 30 minutes, roughly corresponding to about 20 million photons.

As will be described in the Examples, the dual capacity for enhanced specificity and safety was demonstrated both in vitro and in vivo. Thus, making GNS an ideal photothermal transducer for cancer therapy at the nanoscale level. GNS amplifies the thermal conductivity profile of LITT in a manner that conforms to the GNS distribution, reduces procedure duration, and protects surrounding structures. Additionally, GNS selectively accumulates in tumors in an in vivo murine model of primary malignant gliomas.

Nanoplasmonics-Enhanced Laser Interstitial Thermal Therapy (NPE-LITT) includes several important and unique mechanisms. Increased absorption of the excitation light by the plasmonic metal nanoplatforms (i.e., nanostars) resulting in enhanced absorption of the plasmonic metal nanoplatforms. Increased absorption of the excitation light by the plasmonic metal nanoplatforms (i.e., nanostars), resulting in increased heating of the plasmonic metal nanoplatforms (i.e., nanostars). Plasmonic metal nanoplatforms serve as plasmonics-active photon-heat enhancers to extend the treatment zone. Plasmonic metal nanoplatforms can both expand coverage and protect surrounding health tissue structures. Plasmonic metal nanoplatforms selectively accumulate within intracranial tumors due to the EPR effect, resulting in a heating process that adequately conforms to tumor margins. Plasmonic metal nanoplatforms are non-toxic and biocompatible materials suitable for in vivo applications.

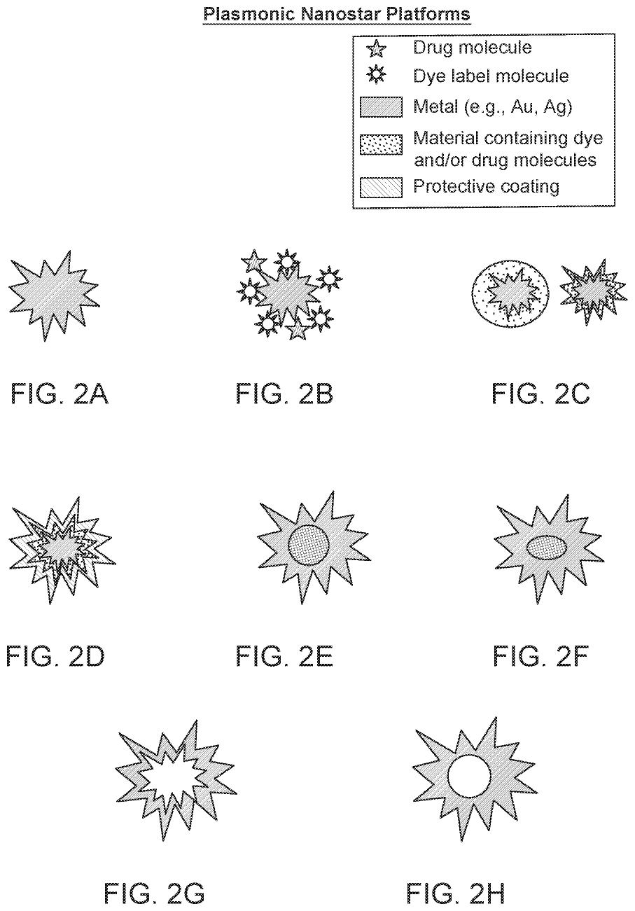

Plasmonic metal nanoplatforms are available in various shapes. Gold nanostars are one embodiment of plasmonic metal nanoplatforms. Nanostars are available in multiple embodiments, as well. FIG. 2 provides a non-exclusive schematic representation of various nanostar embodiments. FIG. 2A shows a plasmonics-active metal nanostar. FIG. 2B shows a nanostar labeled with drug and dye molecules. FIG. 2C shows a nanostar with a layer, embedded with a label and/or a drug. FIG. 2D shows a nanostar with a layer, embedded with a label and/or a drug and a protective layer. FIG. 2E shows a nanostar with a paramagnetic spherical nucleus. FIG. 2F shows a nanostar with an elongated paramagnetic nucleus. In embodiments, the paramagnetic nucleus can be helpful in accumulating the plasmonic metal nanoplatforms in the lesion or tumor. For example, a doctor or surgeon can use a magnetic device to encourage accumulation of plasmonic metal nanoplatforms with paramagnetic nuclei in the site intended for effective treatment (e.g., lesion or tumor to be treated or ablated). FIG. 2G shows a void space nanostar. FIG. 2H shows a nanostar with an empty or dielectric core.

Plasmonic metal nanoplatforms can also be modified with bioreceptors. Bioreceptors can be used to target disease cells or mutate genes or specific biomarkers with exquisite specificity. They can be used to bind a biotarget of interest to a drug system for therapy. Bioreceptors can take many forms. Available bioreceptors are as numerous as the different analytes that have been monitored using biosensors. However, bioreceptors can generally be classified into five different major categories. These categories include: 1) antibody/antigen, 2) enzymes, 3) nucleic acids/DNA, 4) cellular structures/cells and 5) biomimetic (aptamers, peptides, etc).

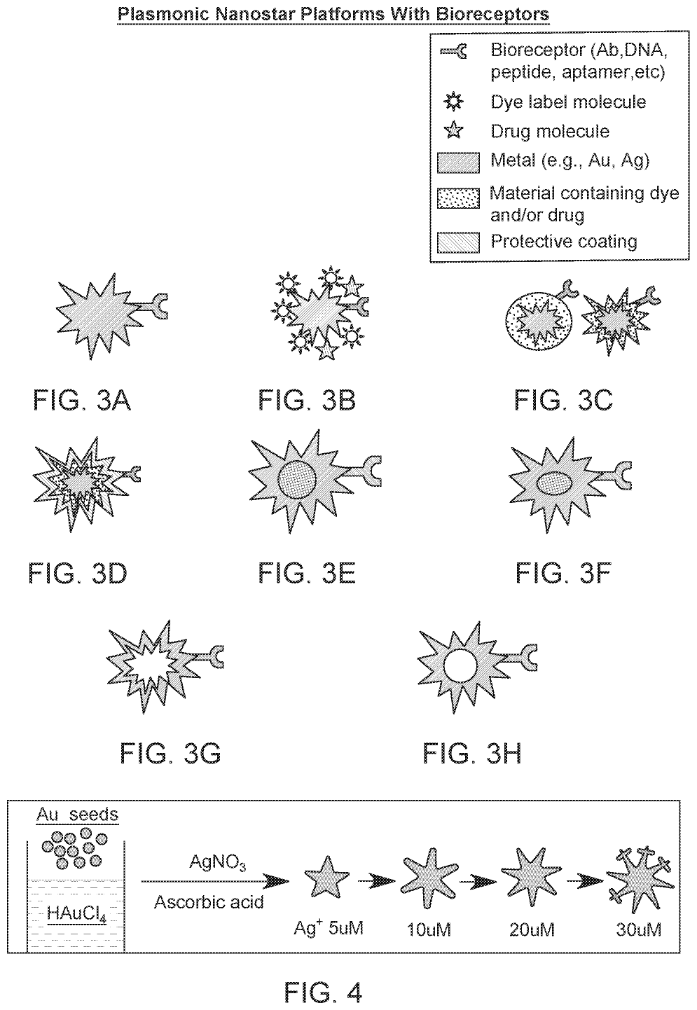

FIG. 3 provides a non-exclusive schematic representation of various nanostars with bioreceptors. The nanostars are similar to those in FIG. 2 but also have a bioreceptor for tumor targeting. FIG. 3A shows a plasmonics-active metal nanostar with a bioreceptor. FIG. 3B shows a nanostar labeled with optical dye and/or drug molecules with bioreceptor. FIG. 3C shows nanostars with layer (embedded with label and/or drug) with bioreceptor. FIG. 3D shows a nanostar with layer (embedded with label and/or drug) and protective overlayer with bioreceptor. FIG. 3E shows a nanostar with paramagnetic spherical nucleus with bioreceptor. FIG. 3F shows a nanostar with elongated paramagnetic nucleus with bioreceptor. FIG. 3G shows a void-space nanostars with bioreceptor. FIG. 3H shows a nanostar with empty or dielectric core with bioreceptor. To specifically target diseased cells, specific genes, or protein markers, the NPE-LITT systems can be bound to a bioreceptor (e.g., antibody, DNA, proteins, cell-surface receptors, aptamers, etc.).

Biomolecules (PA molecules, drugs, proteins, enzymes, antibodies, DNA, etc.) can be immobilized to a solid support, such as a metal nanoparticle, using a wide variety of methods. Binding performed through covalent bonds usually takes advantage of reactive groups such as amine (—NH₂) or sulfide (—SH) that naturally are present or can be incorporated into the biomolecule structure. Amines can react with carboxylic acid or ester moieties in high yield to form stable amide bonds. Thiols can participate in maleimide coupling, yielding stable dialkylsulfides.

Gold and/or silver nanoparticles can be used as a solid support. Many immobilization schemes involving Au (Ag) surfaces utilize a prior derivatization of the surface with alkylthiols, forming stable linkages. Alkylthiols readily form self-assembled monolayers (SAM) onto silver surfaces in micromolar concentrations. The terminus of the alkylthiol chain can be used to bind biomolecules or can be easily modified to do so. The length of the alkylthiol chain has been found to be an important parameter, keeping the biomolecules away from the surface. Furthermore, to avoid direct, non-specific DNA adsorption onto the surface, alkylthiols have been used to block further access to the surface, allowing only covalent immobilization through the linker.

Silver surfaces have been found to exhibit controlled self-assembly kinetics when exposed to dilute ethanolic solutions of alkylthiols. The tilt angle formed between the surface and the hydrocarbon tail ranges from 0 to 15°. There is also a larger thiol packing density on silver, when compared to gold. After SAM formation on gold/silver nanoparticles, alkylthiols can be covalently coupled to biomolecules. Most synthetic techniques for the covalent immobilization of biomolecules utilize free amine groups of a polypeptide (enzymes, antibodies, antigens, etc.) or of amino-labeled DNA strands, to react with a carboxylic acid moiety forming amide bonds. As a general rule, a more active intermediate (labile ester) is first formed with the carboxylic acid moiety and in a later stage reacts with the free amine, increasing the coupling yield.

A gold nanostar not having a chemical or polymer coating can be used as a plasmonics enhanced nanoparticle. The plasmon of the star shaped gold nanoparticle (“nanostars”) can be tuned to the NIR region. Moreover, the structure contains multiple sharp tips that can enhance incident electromagnetic fields. Additionally, it is recognized in the field that NIR-absorbing nanorods, nanocages or nanoshells can be used as contrast agents in optical imaging techniques, such as optical coherent tomography, two-photon luminescence (TPL) microscopy, and photoacoustic imaging. Their large absorption cross-sections can also effectively convert photon energy to heat during photothermal therapy. Nanostars, which absorb in the NIR, are hypothesized to behave similarly. However, nanostar-related bioapplications remain scarce despite their potential, mostly due to the difficulty of surface functionalization.

A seed-mediated, polymer-free synthesis method to produce a chemical or polymer coating-free gold nanostar is described herein. The method results in high-yield monodisperse gold nanostars with a mean tip-to-tip diameter from 50-70 nm. The coating-free nanostars have plasmon bands tunable in the NIR. As discussed above, it is efficient to excite plasmons in the NIR (700-900 nm) for deep tissue penetration of the excitation light.

In embodiments, citrate can be used for stabilization, which simplifies surface modification for further applications. The optical properties and plasmonic tunability of the coating-free nanostars were experimentally examined and compared to polarization-averaged 3-D finite element method (FEM) simulation results. Because laser light is polarized, calculations were performed based on an average of different polarization directions.

Finally, as will be described more fully in the Examples, use of coating-free nanostars as a strong multiphoton contrast agent during in vitro cellular imaging was investigated.

In an exemplary embodiment, nanostars having anisotropically grown gold branches were produced by reducing tetrachloroauric acid onto 12-nm citrate-stabilized gold seeds in an acidic environment using a weak reducing agent, ascorbic acid (AA), and stabilizing with sodium citrate. FIG. 4 is a schematic illustration of the synthesis process. Advantageously, the synthesis is rapid, reproducible and does not require a polymer as surfactant. Unlike previous methods, which take more than hours of synthesis, the growth of nanostars using the seed-mediated, polymer-free synthesis method is completed in less than half a minute. The produced nanoparticles are stable at 4° C. for at least a week after centrifugal washing. The seed-mediated, polymer-free synthesis method is the simplest and quickest nanostar synthesis to date. The polymer-free synthesis method effectively simplifies surface functionalization of nanostars.

Methods to synthesize nanostars of different geometry while keeping the particle size in a similar range were investigated. Multiple factors, including pH, vortexing speed, and concentration of AgNO₃, AA, HAuCl₄ and seed were varied and studied. In general, nanostars formed the most red-shift plasmon under lower pH, higher vortexing speed and AA/HAuCl₄ ratios of 1.5˜2. Concentrations of HAuCl₄ and seeds were selected so the resulting nanostar sizes were around 60 nm.

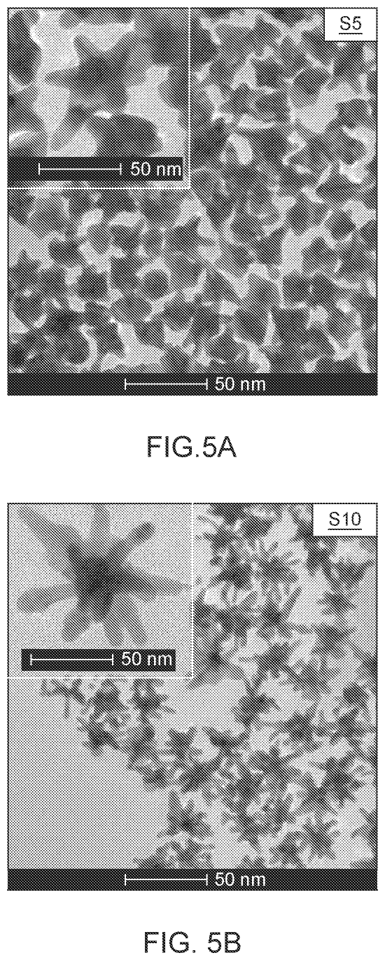

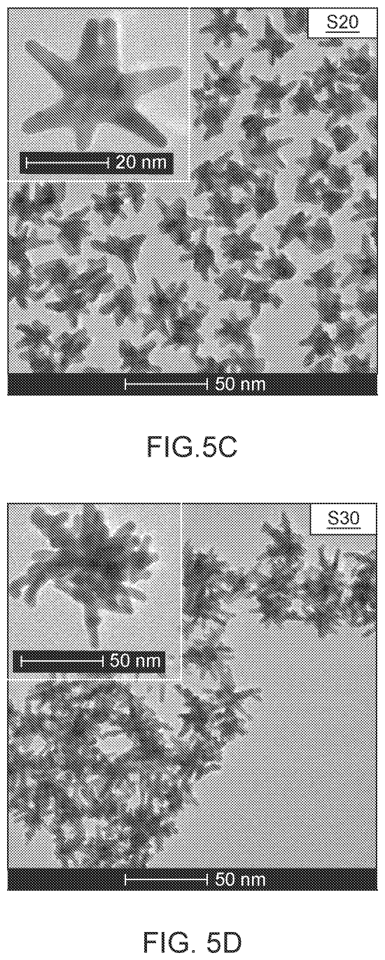

Importantly it was determined that silver ions play a major role in controlling the formation of the star geometry. If Ag⁺ were not added during synthesis, the resulting particles were polydisperse in both size and shape. Adding a relatively small amount of Ag⁺ led to high-yield monodisperse star-shaped particles. The overall particle diameters synthesized under different Ag⁺ concentrations were within 50˜70 nm. Under higher Ag⁺ concentrations, sharper and more numerous branches were formed. For example, Ag+ concentrations higher than 20 μM led to high-yield monodisperse star-shaped particles. FIG. 5 includes a series of TEM images of nanostars formed under different silver ion concentrations. The nanostars in FIG. 5 S5 were formed using an Ag⁺ concentration of 5 μM. The nanostars in FIG. 5 S10 were formed using an Ag⁺ concentration of 10 μM. The nanostars in FIG. 5 S20 were formed using an Ag⁺ concentration of 20 μM. The nanostars in FIG. 5 S30 were formed using an Ag⁺ concentration of 30 PM. While not being bound by theory, it is believed that the major role of Ag⁺ is not to form Ag branches but to assist the anisotropic growth of Au branches on multi-twinned citrate seeds, but not single crystalline CTAB seeds.

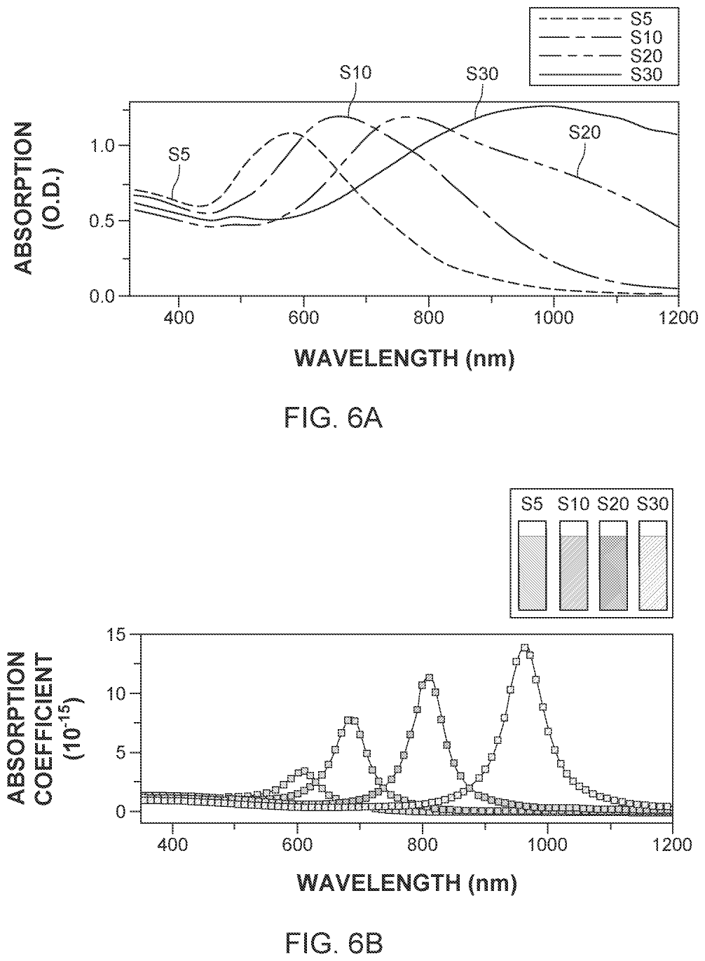

Plasmon tunability was achieved by adjusting the Ag⁺ concentration during synthesis. Specifically, higher concentrations of Ag⁺ progressively red-shifted the plasmon band. As can be seen in the TEM images in FIG. 5, higher Ag⁺ concentrations lead to the formation of longer, sharper, and more numerous branches. As shown, the nanostars in FIG. 5 S5 had a few protrusions, while the nanostars in FIG. 5 S30 had multiple long, sharp branches. The overall size of all sample nanostars was less than 100 nm, which is smaller than previously reported nanostars. FIG. 6 includes two charts: 6A and 6B. FIG. 6A shows absorbance spectra (unnormalized) of the star solution (˜2 nM) in citrate buffer versus wavelength for the different Ag⁺ concentrations. FIG. 6B shows FEM generated absorption spectra of nanostars embedded in water versus wavelength for the different Ag⁺ concentrations. The solved data points were interpolated with a spline fit. The orientation dependence of the incident E-field was accounted for by averaging the absorption spectra of the nanostars as they were incrementally rotated by 30 degrees in the [x=y] plane, such that the orientation of the branches relative to the z-polarized incident field became randomized.

FIG. 6 illustrates that the plasmon peak of nanostars is tunable from 600 nm to 1000 nm by adjusting the Ag⁺ concentration. Additionally, it was determined that the solution color changed from dark blue to dark grey as the plasmon red-shifted and broadened. Both the plasmon peak position and spectral width (as defined by the full width at half maximum (FWHM) of the plasmon peak) followed a linear trend with increasing Ag⁺ concentration. A plateau was reached around an Ag⁺ concentration of 30 μM. This testing showed that nanostars can be synthesized in a controlled fashion and used as potential candidates for NIR applications.

As mentioned above, the thermal response of laser-irradiated tissue is highly dependent on the unique optical properties of the tissue. Photons propagating in the tissue go through a series of scattering and absorption events wherein the photon's energy is randomly scattered off in a different direction or absorbed. The behavior of the photon in tissue is thus highly dependent on the molecular composition and geometrical configuration of the tissue.

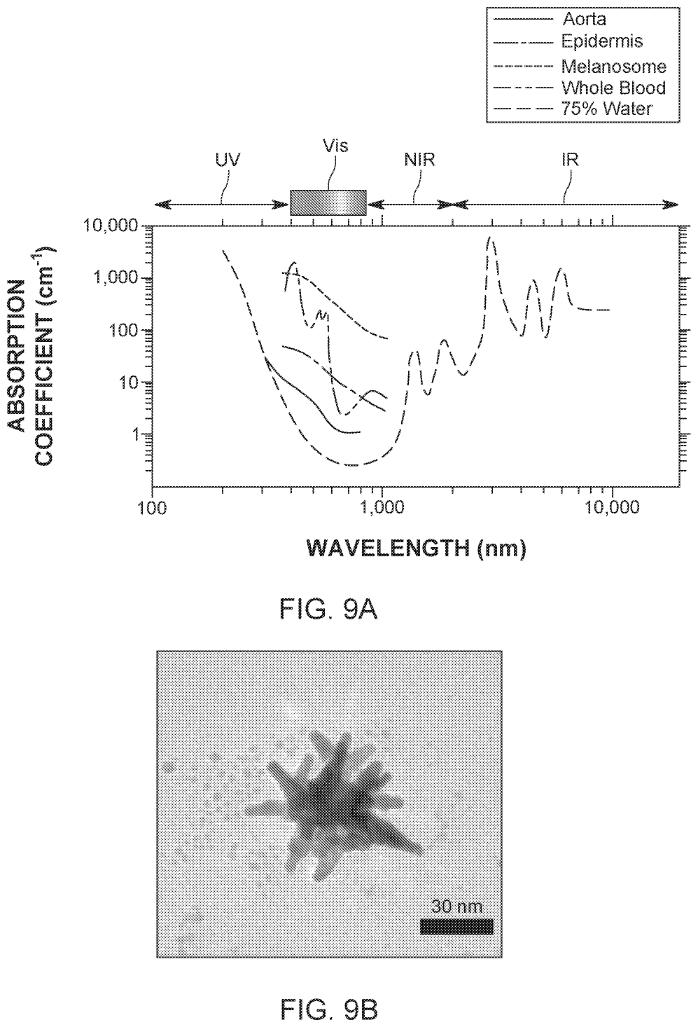

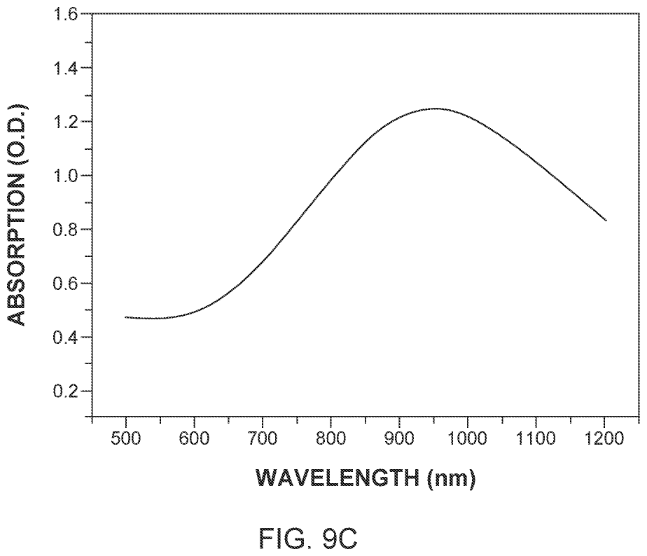

Advantageously, gold nanostars can be synthesized in a controlled fashion and exploited as a ‘photothermal adjuvant’ for LITT excitation in the tissue “optical window”, where photons travel further in healthy tissue to be ‘captured’ and converted into heat by GNS within a cancer. Within the optical tissue window, most tissues are sufficiently weak absorbers to permit significant penetration of light. This optical window ranges from 600 to 1300 nm. FIG. 9A is a chart showing wavelengths in the optical window of tissue and absorption spectra of biological components.

At the short-wavelength end, the window is bound by the absorption of hemoglobin, in both its oxygenated and deoxygenated forms. At shorter wavelengths the absorption of many more biomolecules in tissue becomes important, including DNA and the amino acids tryptophan and tyrosine. At the longer wavelengths (infrared end) of the window, light penetration is limited by the absorption properties of water. Within the “therapeutic window”, scattering is dominant over absorption, and so the propagating light becomes diffuse, although not necessarily entering the diffusion limit.

GNS that can produce efficient photothermal effects around 1064-nm laser wavelength have been produced and are described more fully herein. The GNS were synthesized using a modified approach based on the surfactant-free method. In the method, 12-nm gold sphere nanoparticles synthesized by reducing HAuCl4 with trisodium citrate were used as seeds, and subsequently were rapidly mixed with AgNO₃, ascorbic acid and HauCl4. The ratio between seeds and HauCl4 or AgNO3 was tuned to achieve high absorption at 1064 nm. The synthesized GNS were coated with SH-mPEG (M.W. 5000) to improve in vivo stability and circulation time. PEG-functionalized GNS nanoparticles were condensed, and the gold mass concentration was measured with inductively coupled plasma mass spectrometry (ICP-MS) using Varian 820 mass spectrometer (Varian, Palo Alto, CA, USA). The GNS concentration was measured with atomic absorption spectroscopy (AAS). FIGS. 9B and 9C show the transmission emission microscopy (TEM) image and the absorption spectrum of GNS engineered to have absorption around 1064 nm within the tissue window.

The method described herein can be used to enhance absorption of excitation light from laser therapy in a lesion or tumor being treated with said laser therapy. The method includes administering plasmonic metal nanoplatforms to a subject having the lesion or tumor to be treated with laser therapy. The plasmonic metal nanoplatforms selectively accumulate in the lesion or tumor, which may have different and/or irregular shapes. The plasmonic metal nanoplatforms fill the contours of the lesion or tumor to reflect the shape of the lesion or tumor. The lesion or tumor is treated in a conformal way with laser therapy in which the excitation light from the laser therapy is strongly absorbed by the plasmonic metal nanoplatforms accumulated in the lesion or tumor thereby filling contours of the lesion or tumor.

The method described herein can be used to enhance absorption of excitation light from laser therapy in a lesion or tumor being treated with said laser therapy. The method includes administering plasmonic metal nanoplatforms to a subject having the lesion or tumor to be treated with laser therapy. The plasmonic metal nanoplatforms selectively accumulate in the lesion or tumor. The lesion or tumor is then treated with laser therapy and the excitation light from the laser therapy is absorbed by the plasmonic metal nanoplatforms accumulated in the lesion or tumor thus inducing photon-to-heat conversion in the plasmonic metal nanoplatforms, thus transforming said nanoplatforms into heat sources, leading to further heat propagation in tissue, lesion or tumor and thereby inducing a larger treatment area.

### EXAMPLES

### Example 1

The ability of GNS to selectively accumulate in brain tumor tissue was evaluated. A GNS nanoprobe radiolabeled with ¹²⁴I for sensitive brain tumor detection using PET imaging was prepared. GNS nanoparticles were labeled with ¹²⁴I through strong I—Au chemical bonding with >98% labeling efficiency after 30 minutes incubation at room temperature. The stability of radiolabeled GNS was examined in both phosphate-buffered saline (PBS) and plasma with anti-clotting heparin. Experimental results showed that 97.2±0.2% (PBS) and 97.7±0.4% (plasma) of ¹²⁴I remained on the GNS after 7-day incubation at 37° C.

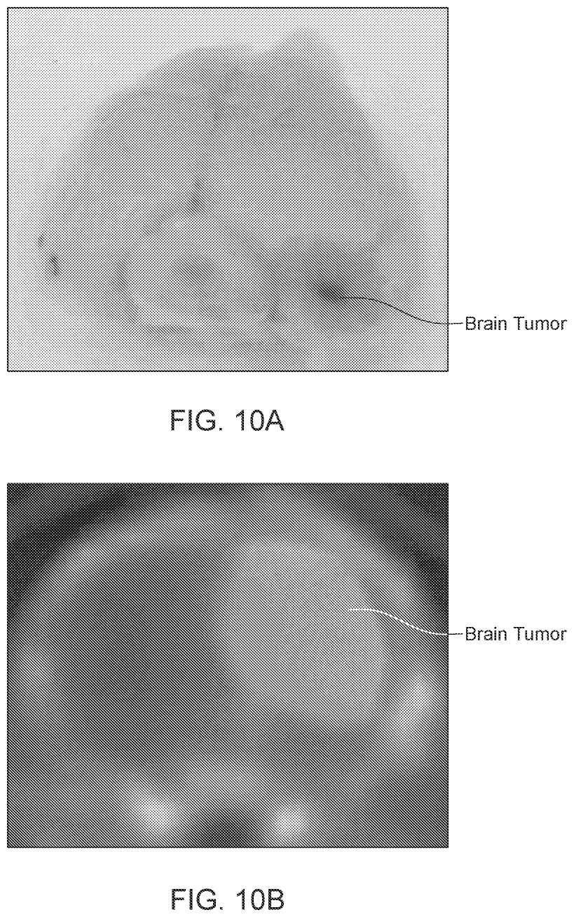

Testing in murine models showed that the GNS nanoprobe accumulated selectively in brain tumors through compromised blood-brain barrier (BBB) (FIG. 10) after injection via tail vein into two different orthotopic glioma models, intracranial injection of U87MG GBM cells (FIG. 10A) and neural stems cells with IDH-1, p53 and PDGFB gene mutations (FIG. 10B).

FIG. 10(A) is a gross specimen of mouse brain with brain tumor shown in black color due to the uptake of light absorbing GNS nanoparticles. The specimen was collected 24 hours after IV injection of GNS. FIG. 10(B) is a PET/CT scan of brain-tumor bearing mouse 48 hours after 124I labeled GNS IV injection. The average GNS tumor uptake is 7.2% ID/g. The unit (% ID/g) indicates percent injected dose per gram tissue. To determine % ID/g, the tumor weight was measured using an electronic balance. After measuring weight, the tumor was digested with aqua regia, and the Au mass was measured with inductively coupled plasma mass spectrometry (ICP-MS) with a Varian 820 mass spectrometer.

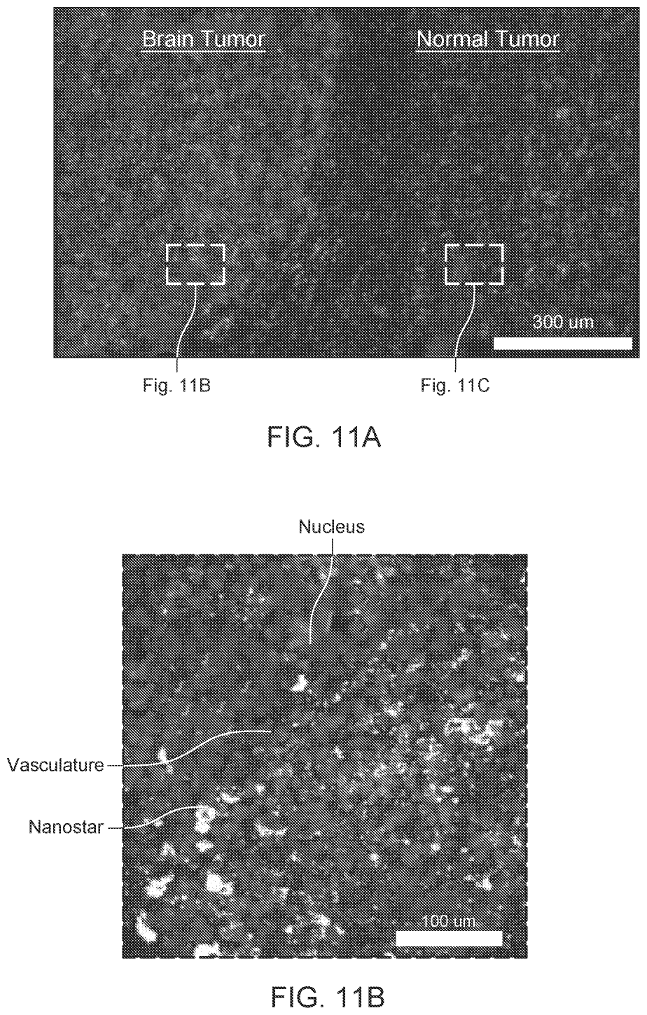

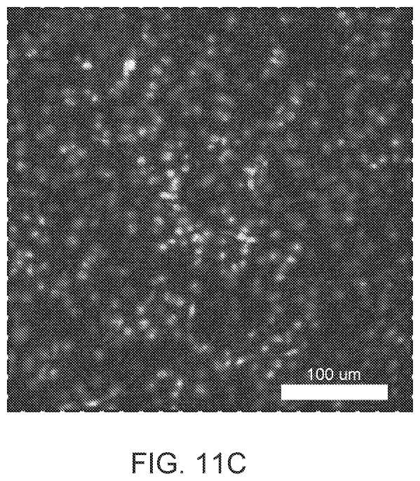

High-resolution two-photon microscopy was used to confirm that GNS nanoparticles accumulate only within the brain tumor boundary but not in surrounding healthy brain tissue after systemic administration (FIG. 11).

FIG. 11 is a set of images showing that GNS nanoprobes selectively accumulate in the brain tumor after IV injection. In the images, GNS (bright spots) only appear in the tumor part but not in the normal brain tissue. There is a clear boundary for GNS distribution between tumor and normal brain tissue. In the images, red indicates vasculature, blue indicates cell nucleus, and white spots indicate GNS. GNS nanoprobes have extremely high two-photon luminescence (TPL) cross-section (50,000 times higher than gold nanospheres) due to tip-enhanced plasmonics.

### Example 2

Laser therapy, which combined GNS with a LITT system was tested. Gold nanostars (GNS) were synthesized. All chemicals were purchased from Sigma-Aldrich (St. Louis, MO) and used directly without further purification. Gold nanostar particles were synthesized using the surfactant-free method described above.

Briefly, 12-nm gold sphere nanoparticles synthesized by reducing HAuCl₄ with trisodium citrate were used as seeds, and subsequently were rapidly mixed with AgNO₃, ascorbic acid and HauCl₄. The ratio between seeds and HauCl₄ or AgNO₃ was tuned to achieve high absorption at 1064 nm. The synthesized GNS were coated with SH-mPEG (M.W. 5000) to improve in vivo stability and circulation time. PEG-functionalized GNS nanoparticles were condensed, and the gold mass concentration was measured with inductively coupled plasma mass spectrometry (ICP-MS) using Varian 820 mass spectrometer (Varian, Palo Alto, CA, USA). The GNS concentration was measured with atomic absorption spectroscopy (AAS). FIGS. 9B and 9C show the transmission emission microscopy (TEM) image and the absorption spectrum of GNS engineered to have absorption around 1064 nm within the tissue window.

Female C57BL/6 mice were used at 6-12 weeks of age for the testing. C57BL/6 mice were purchased from Charles River Laboratories, Wilmington, MA, USA. CT-2A is a syngeneic murine glioma cell line on C57BL/6 background. The CT-2A murine glioma model is considered to accurately represent several glioblastoma (GBM) characteristics including intra-tumoral heterogeneity, in vivo migratory patterns, radio-resistance, chemo-resistance and different modes of immune dysfunction observed in GBM. The cell line was authenticated using the National Institute of Standards and Technology. Interspecies contamination check for human, mouse, rat, African green monkey, and Chinese hamster was also performed. The cell working stocks tested negative for Mycoplasma spp. and karyotyped. CT-2A is not among the ICLAC database of commonly misidentified cell lines. The CellCheck Mouse Plus cell line authentication and Mycoplasma spp. testing services were provided by IDEXX Laboratories (Westbrook, ME, USA). CT-2A tumor cells were grown in vitro in Dulbecco's Modified Eagle's Medium (DMEM) with 2 mM 1-glutamine and 4.5 mg/mL glucose (Thermo Fisher Scientific, Waltham, MA, USA) containing 10% fetal bovine serum (FBS) (Gemini Bio-Products, Sacramento, CA, USA). Cells were harvested in the logarithmic growth phase. For intracranial implantation, tumor cells in phosphate buffered saline (PBS) were then mixed 1:1 with 3% methylcellulose and loaded into a 250-μl syringe (Hamilton, Reno, NV). The needle was positioned 2 mm to the right of the bregma and 4 mm below the surface of the skull at the coronal suture using a stereotactic frame. Then, 1×10⁴ tumor cells were delivered in a total volume of 5 μL per mouse. Animals were maintained under specific pathogen-free conditions at the Cancer Center Isolation Facility of Duke University Medical Center. All experimental procedures were approved by the Institutional Animal Care and Use Committee.

PET/CT imaging was performed using a small animal PET/CT scanner (Model Siemens Inveon, Siemens Medical Systems, Knoxville, TN, USA). Four mice with brain tumors and one without were injected with gold nanostars (˜100 μCi) through the tail vein. 10-minute PET scans were started immediately after injection, followed by a CT scan. Follow-up PET/CT scans were obtained at 24-, and 72-hours post injection. Mouse brain was harvested after PET/CT scan for histopathology and autoradiography imaging. H&E stain imaging was performed using an Axio Imager widefield microscope (Carl Zeiss, Oberkochen, Germany) coupled with an Axiocam 506 color camera.

Two methods of simulating laser therapy with a LITT system in a subject having a tumor were used to evaluate and study using the LITT system as it is conventionally used versus using the LITT system in conjunction with gold nanostars. One simulation method was a computer simulation, and the other simulation method used tissue phantoms to simulate heat transfer through tissue. The results were used to characterize the attenuation and penetration depth of the LITT system.

The computer simulation was performed using mcxyz.c, which is a computer simulation of the distribution of light in a complex tissue that includes many different types of tissues, each with its own optical properties. The software uses the Monte Carlo method of sampling probabilities for the stepsize of photon movement between scattering events and for the angles (θ,ψ) of photon scattering.

Different tissue types (including the tumor and surrounding white and gray matter) have vastly different optical characteristics. Thus, to match the experimental parameters of the laser therapy experiments, the optical properties of the tissue in the simulation software were set to that of gray matter and the light source was set as a single point source located at the interface between water and the tissue. A simulation was created to mimic a 0.1-nM concentration of GNS within the tissue to show the increased absorption of photons around a volume of GNS. For the second simulation, the laser source was set to be a point source centered at the interface of water and gray matter to model the contact between the optical fiber of the LITT system and the brain tissue of the LITT procedure. To match the typical amount of gold nanostar found accumulated in tumors, the concentration of the GNS particles was set to 0.1 nM, which corresponds to an absorption coefficient of about 2 cm⁻¹. The particle was also assumed to have little scattering, so the scattering coefficient was set to 1 cm⁻¹. A collimated beam with a radius of 1 cm and an isotropic point source placed 3 cm within the volume were chosen as sources. These correspond to typical laser irradiation configurations with a standoff beam and a fiber delivered source. A spherical volume containing only the gold nanostar was embedded within the tissue near the source. Each simulation was set to run for 30 minutes, roughly corresponding to about 20 million photons.

Optical tissue phantoms were used to simulate the diffusion of light as it travels through tissue. An agarose-based gel (2-3 w/v %) was used because it can serve as a solid scaffold for the gold nanostars (GNS) as well as mimic the heat transfer in tissue due to its high water composition. For the initial LITT heating experiments, a 12×12×10 cm solid gel phantom containing a smaller 5×5×5 cm gel cube infused with GNS at a 0.1 picomolar concentration was prepared to simulate a tumor within normal tissue. An identical phantom without GNS was also produced. A third model was generated with a cylindrical shape (radius of 2 cm) containing GNS in half of the phantom. Phantoms were maintained at room temperature immediately prior to LITT administration.

For all MRI-guided laser interstitial thermal therapy (LITT) for the phantom tissue samples, a NeuroBlate System (Monteris Medical Corporation, Plymouth, MN, USA) was used. The NeuroBlate system includes a sapphire capsule with an internal cooling mechanism using CO₂ gas. The catheter for the NeuroBlate system for this experiment had a diameter of 3.2 mm. The NeuroBlate system transmited pulsed laser light at a wavelength of 1064 nm to thermally ablate target tissue in both the gel phantom with the presence of GNS and the gel phantom without GNS. Heating information was measured using M-Vision, which is the proprietary software that accompanies the NeuroBlate System for planning, executing, and measuring the controlled heating. M-Vision was used to determine the time and temperature of randomly selected “pick points” throughout the ablative field for both sample groups (with GNS and without GNS). As the MRI system measured relative temperature changes, temperatures were reported as the change from baseline. Temperatures were measured and recorded every 7 seconds and the laser was activated for a total time of at least 12 minutes for each experiment.

An IMRIS® (Minnetonka, MN, USA) intra-operative MRI system with a 3.0 Tesla Siemens (Erlangen, Germany) magnet was used for imaging. For LITT, a volumetric rapid gradient-echo (MP RAGE) T1-weighted sequence was utilized throughout the experiment. During treatment, the M-Vision software displayed three, 5-mm thick magnetic resonance slices that were perpendicular to the laser probe trajectory, including: the current treatment slice, one slice deeper, and one slice more superficial with no gap between slices, providing an overall visual coverage of 15 mm in thickness perpendicular to the probe. This data provided an estimate of thermal expansion in the 3D volume. The software also displayed a single coronal image and a single sagittal image, which were updated in real-time throughout the procedure to show the cumulative treatment effect as heating progressed. After the probe was set to the chosen starting depth, quantitative MRI-based temperature mapping based on the proton resonant frequency shift sequences (i.e., MR thermography) were started. At least eight cycles (each lasting 7 seconds) of baseline scanning were completed prior to firing the laser per the NeuroBlate System protocol.

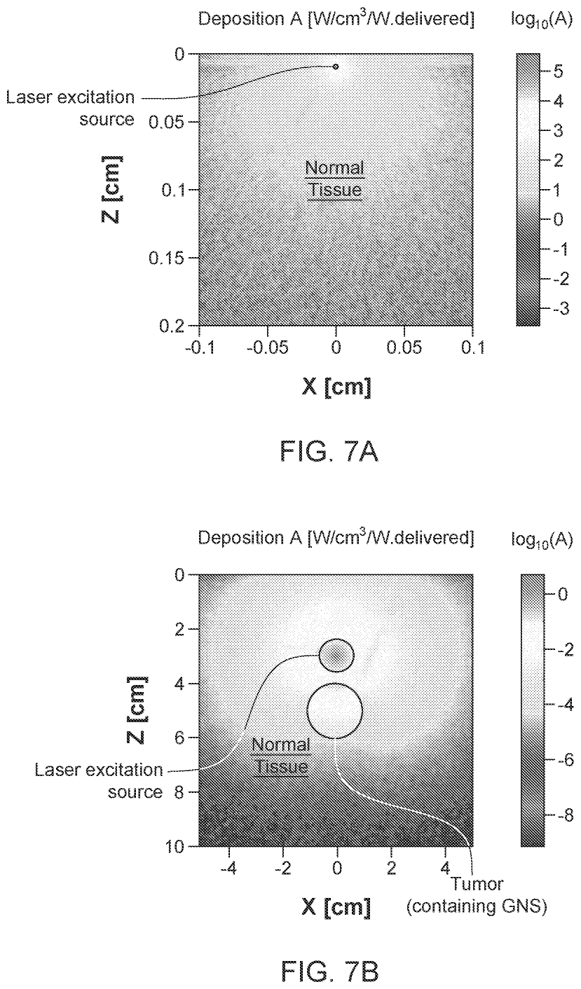

A computer simulation of a LITT heated tumor model containing gold nanostars was studied. FIG. 7A shows the numerical simulation of an idealized tumor model wherein gold nanostars are localized in a region analogous to gold nanostars selectively accumulated in tumors due to the EPR effect. Additionally, the optical properties of the tissues in this model were selected to mimic the properties of actual tissue (i.e., gray tissue of brain matter). The results show the logarithmic absorption of energy through gray matter tissue near the laser source. As seen in FIG. 7A, most of the energy was deposited in a localized region close to the laser source and quickly dropped off after a few millimeters. The results of the phantom tissue simulation for the tissue with GNS are shown in FIG. 7B. The results show that plasmonic GNSs contained in tumors can both expand coverage and protect surrounding healthy tissue structures.

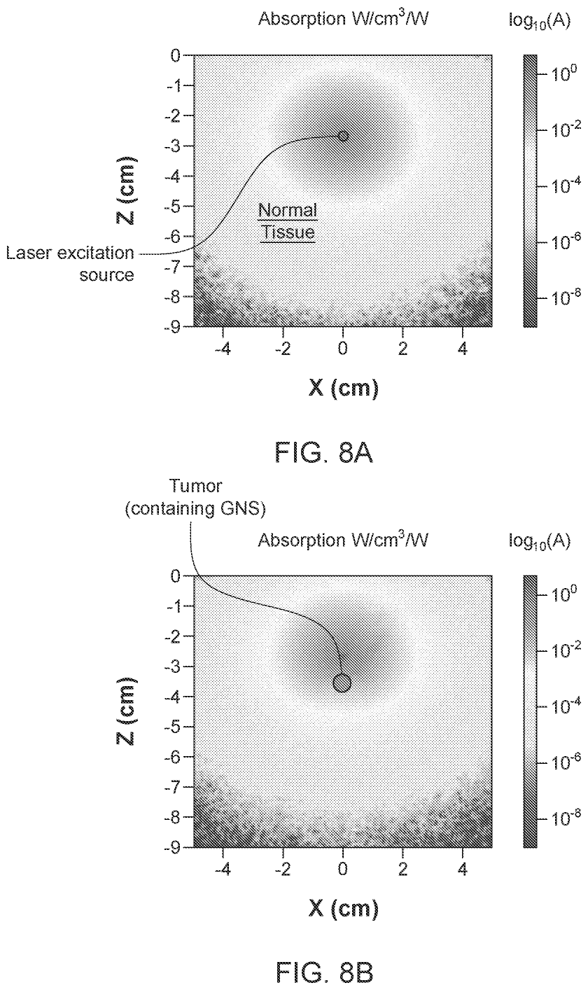

FIG. 8A is a normalized energy absorption map measured in the area around the isotropic point source within the layer mimicking the optical response of gray matter brain tissue. FIG. 8B shows the energy absorption map of a simulated tumor (the spherical volume of gold nanostars) near the excitation laser point source. The Monte Carlo theoretical simulation results show markedly higher absorption of the tumor area relative to the surrounding tissue. The results of these simulations point to higher specificity in photon absorption where there are gold nanostars. With gold nanostars' high photon-to-heat conversion, heating is much more efficient as well. This demonstrates the feasibility of gold nanostars as a nanoplatform for selective and efficient heating of targeted photothermal therapy.

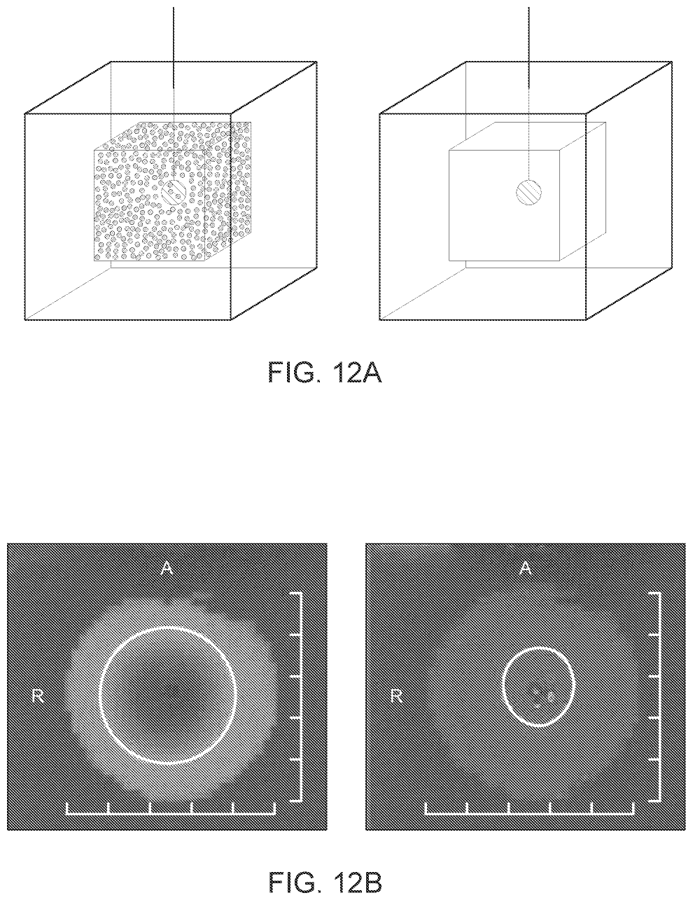

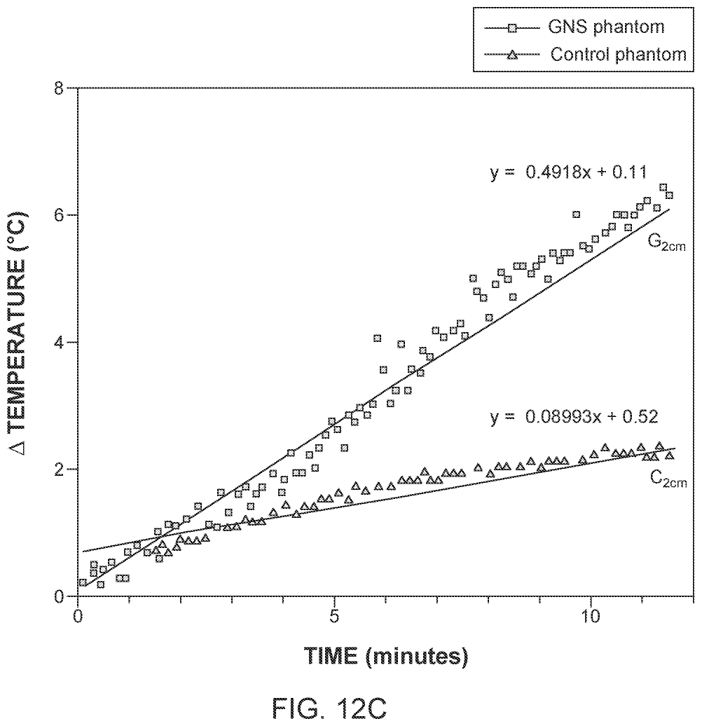

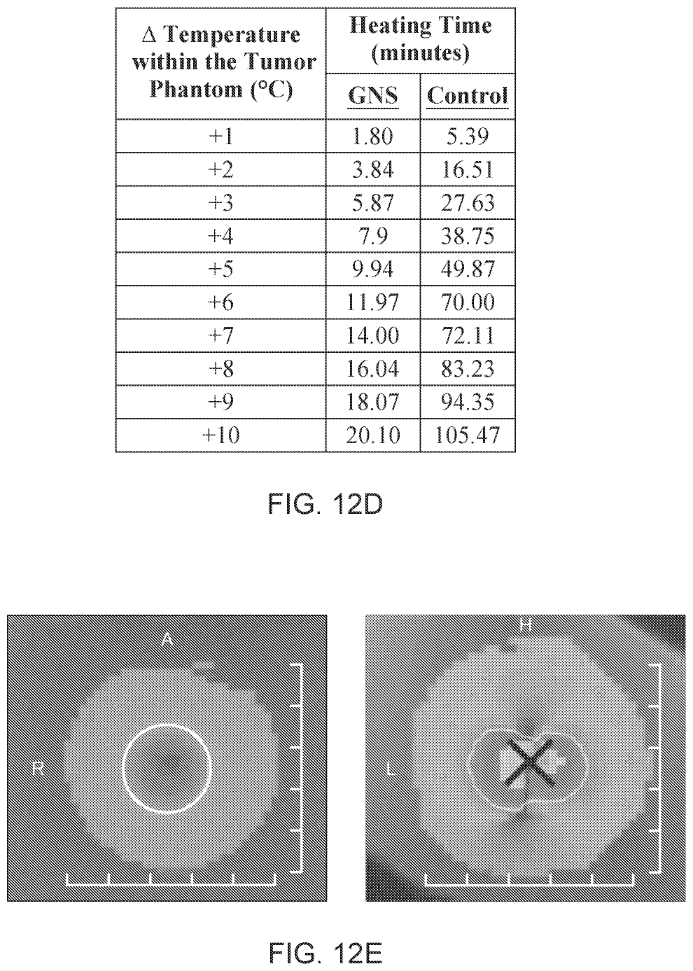

As described above, the differential conduction of thermal energy across tumor and normal brain tissue creates a limitation on the current applications of LITT. The phantom tissue samples and NeuroBlate system described in Example X were used to evaluate how GNS infusion impacted thermal treatment efficiency and accuracy for different lesion or tumor conformations. FIG. 12A shows a diagram of the phantom tumor models. In FIG. 12, the external cube is a 12×12×10 cm solid agarose gel, and the internal cube is a 5×5×5 cm solid agarose gel. The image on the left includes embedded gold nanostars and the image on the right does not. Temperatures were measured at a point 2 cm from the laser tip (within the smaller cube of each phantom). Representative images from thermal monitoring after 12 minutes of heating are shown in FIG. 12B. The image on the left is for the embodiment with gold nanostars in the internal cube, and the image on the right is for the embodiment with no gold nanostars. The marked line represents tissue exposed to the thermal equivalent of 43° C. for at least 2 minutes.

FIG. 12C is a graph of temperatures measured 2 cm from the laser probe tip in the gold nanostar infused model (GNS phantom) and control (Control phantom) during the administration of laser interstitial thermal therapy (LITT). Equations for simple linear regressions are shown in the figure. As can be seen in FIG. 12C, the GNS-infused phantom grossly demonstrated an increased rate of heating. A simple linear regression model fitted to the data set showed that the rate of temperature increase for the GNS phantom was nearly 5.5 times greater than that of the control. FIG. 12D is a table showing extrapolated and interpolated heating times and temperatures within the tumor phantom (2 cm from the probe tip) using the simple linear regressions calculated from the data in FIG. 12C. The regression lines both demonstrated a high degree of accuracy, with R-squared values of 0.9743 for the GNS phantom and 0.9152 for the control phantom, respectively. These results support the hypothesis that GNS infusion leads to more efficient conduction of thermal energy and subsequently more rapid increases in temperature to ablative thresholds.

Further testing was performed to evaluate whether the shape of the GNS distribution influenced the pattern of heating in the phantom model system. The experiment described above was repeated with the internal agarose-gel having a star shape rather than a cube shape (as was used in the initial experiment). Thus, the GNS was infused into a star-shaped internal phantom. FIG. 12E includes representative images from the M-Vision MRI-thermometry software for the cube and star shaped internal phantoms. The top image is for the cube shaped internal phantom and the bottom image is for the star shaped internal phantom. The adjusted contouring of the heated region provides evidence that the distribution of heat follows the distribution of GNS within the tumor model. The marked line represents tissue exposed to the equivalent of 43° C. for at least 2 minutes.

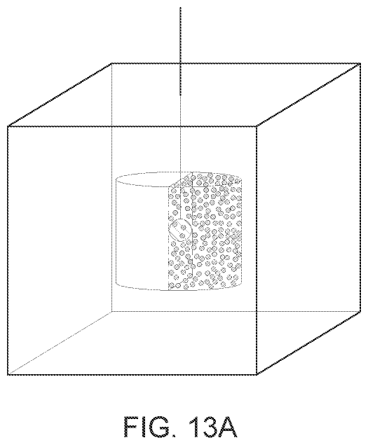

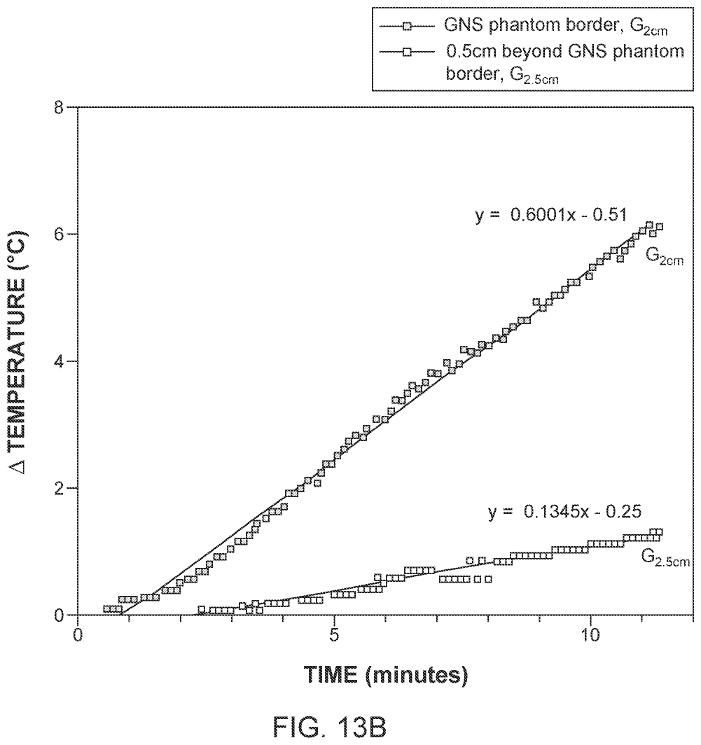

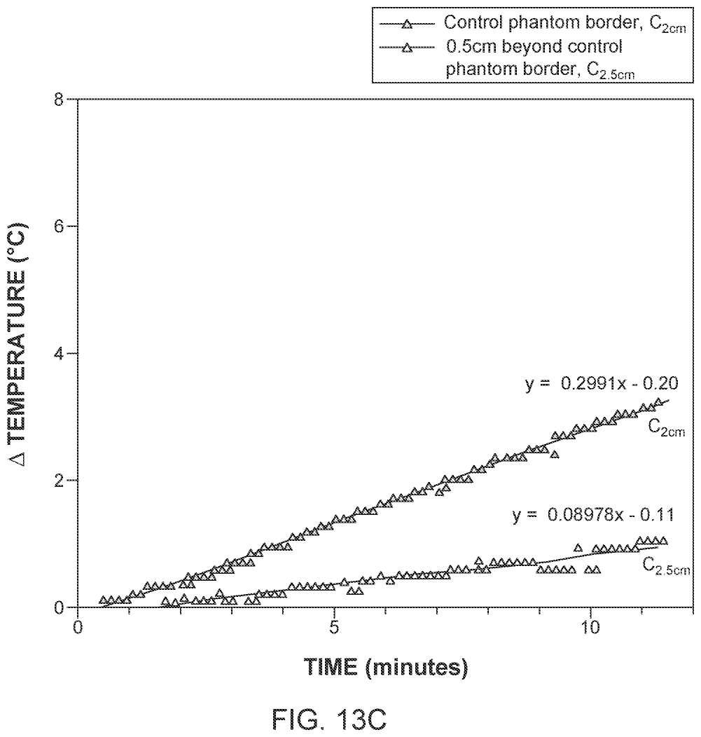

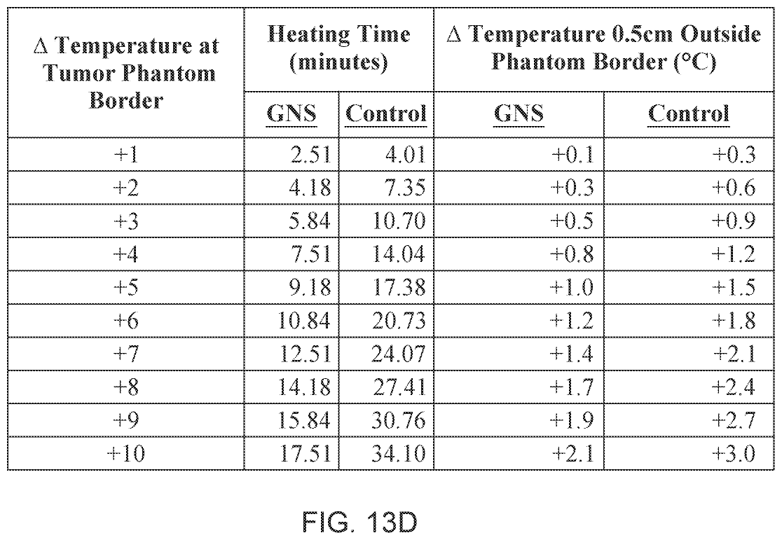

Testing was performed to evaluate whether GNS infusion affected the temperature that tissue surrounding a tumor was exposed to during LITT administration. Another tumor phantom was produced with an internal cylinder half infused with GNS. FIG. 13A is a schematic image illustrating the phantom prepared for this experiment. The external cube was a 12×12×10 cm solid agarose gel. The internal cylinder had a radius of 2 cm with half the cylinder containing gold nanostars (GNS).

LITT was administered per the methods described in Examples X above. Temperatures were monitored at the border of the tumor phantom (2 cm from the laser probe, G/C2 cm), and at a point 0.5 cm beyond the border (2.5 cm total from the laser probe, G/C2.5 cm) in an area representing normal surrounding tissue.

FIG. 13B is a graph of temperatures measured in the split phantom model half infused with gold nanostars (GNS). Temperature was recorded at the tumor phantom border (G/C2 cm) and 0.5 cm beyond the border (G/C2.5 cm) during the administration of laser interstitial thermal therapy (LITT). As can be seen in FIG. 13B, the GNS infused half demonstrated accelerated heating. Simple linear regression models were fitted to each set of data points and plotted. The equations for the simple linear regressions are shown in FIG. 13B. The lines demonstrated a high degree of accuracy, with R-squared values for G2 cm of 0.9949, G2.5 cm 0.9641, C2 cm 0.9965, and C2.5 cm 0.9518.

The regressions were used to interpolate and extrapolate heating times and temperatures for various target temperature changes at the tumor border. The results are shown in the chart in FIG. 13C. As can be seen in FIG. 13C, at each target tumor border temperature change, the GNS infused phantom required less time and exposed surrounding structures to lower levels of heating. For example, to increase the temperature +8° C. at the phantom border, the GNS-infused side required 14.18 minutes of heating compared to 27.41 minutes in the control. Moreover, the point 0.5 cm beyond the border was heated to +1.7° C. and +2.4° C., respectively. These differences indicate a protective effect of GNS on surrounding structures by reducing their exposure to potentially damaging thermal energy. While not being bound by theory, it is theorized that this result may be due to the lower levels of photothermal energy needed to raise the temperature of GNS-infused substances.

### Example 3

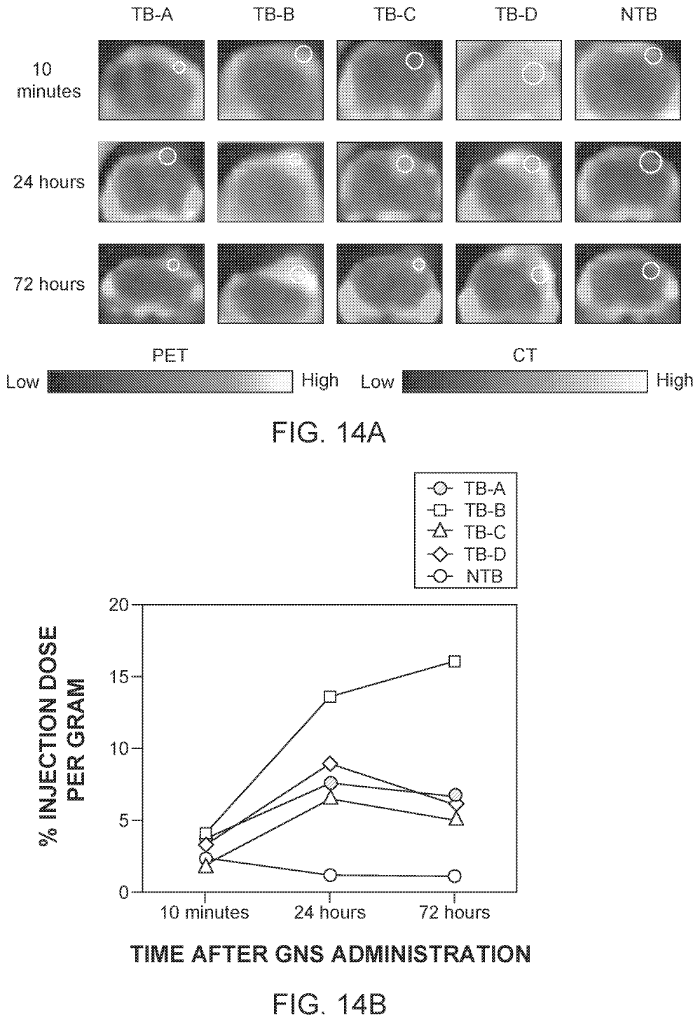

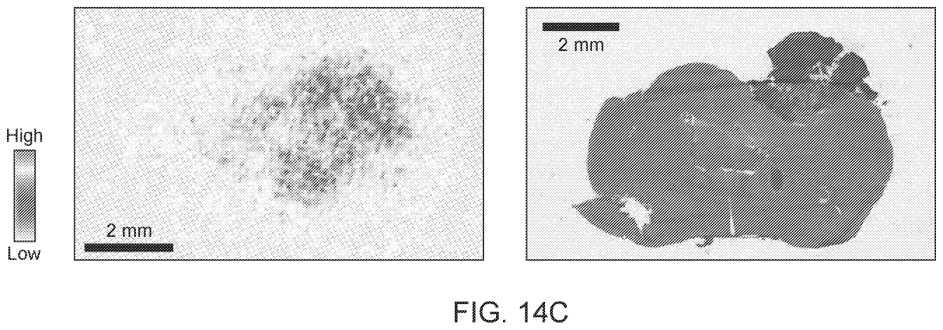

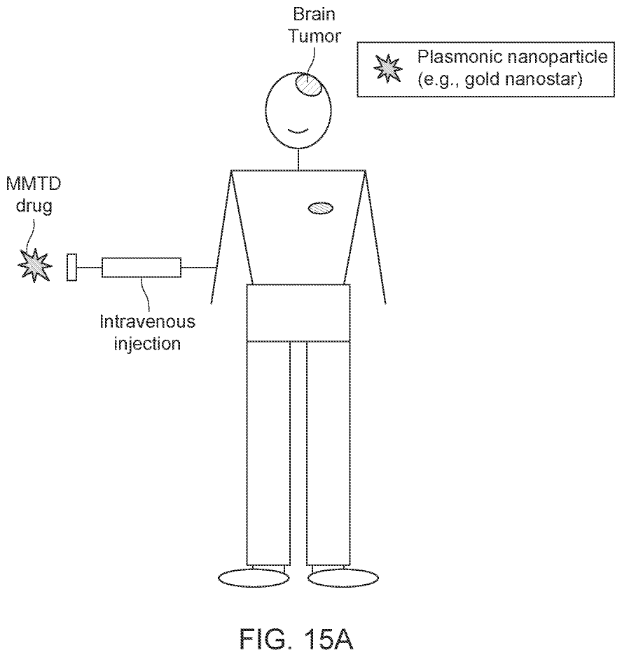

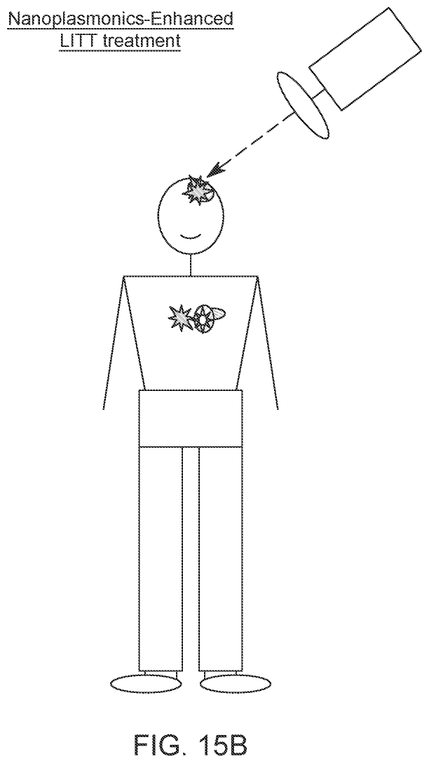

Testing was performed to assess accumulation of GNS in murine brain tumors. PET-CT and autoradiography.xt were used as imaging tools. GNS nanoparticles were labeled with 124I by incubation at room temperature for 10 minutes and then purified by centrifugation wash. The purified 124I radiolabeled GNS nanoparticles were systemically administered into 4 tumor bearing (TB-A, TB-B, TB-C, and TB-D) mice and 1 non-tumor bearing (NTB) mouse via tail vein injection. PET/CT scan was performed at 10 minutes, 24 hours, and 72 hours after the 124I-GNS IV injection. The coronal results are shown in FIG. 14A, which is a coronal PET/CT scan of brain for tumor-bearing (TB-A, TB-B, TB-C, TB-D) and non-tumor-bearing (NTB) mice at 10 minutes, 24 hours, and 72 hours after intravenous injection of 124I-GNS nanoprobes. As can be seen in FIG. 14A, there was no selective uptake of GNS inside the brain in any mice at the 10-minute timepoint. All TB mice demonstrated tumor-selective uptake of GNS at 24 and 72 hours, while the NTB mouse did not show any GNS uptake at any timepoint.

The degree of GNS uptake in the brain was also measured from PET/CT scan. FIG. 14B is a chart showing GNS uptake (% injection dose per gram) in the brain tumor versus time for TB and NTB mice at each given timepoint after GNS administration. As shown in FIG. 14B, GNS uptake in TB mice increased relative to background signal levels for the 10-minute, and the 24- and 72-hour post-administration scans, with TB-A, TB-C, and TB-D reaching their peak uptake at the 24-hour mark while TB-B uptake continued to increase at the 72-hour scan. TB-B demonstrated the highest overall GNS uptake, reaching 16.1% ID/g at the 72-hour timepoint, while the NTB mouse did not exceed the background signal levels at any timepoint.

After the 72-hour scan, the mice were sacrificed, and brain samples removed for further analysis. Radioisotope uptake was assessed and the brains frozen sectioned for autoradiography and after 15 half-lives decay stained with H&E. A representative example of the autoradiography and H&E stain can be seen in FIG. 14C. FIG. 14C provides two images: an autoradiography (left) and H&E histopathology imaging (right) of brain tumor tissue sections from a TB mouse administered intravenous GNS. A high degree of radioactivity was found at the location of the brain tumor. The two images demonstrate overlap between the accumulated radio-emitting particles and the tumor. These results demonstrate that GNS nanoparticles can accumulate selectively inside brain tumors in a murine model system.

One skilled in the art will readily appreciate that the present disclosure is well adapted to carry out the objects and obtain the ends and advantages mentioned, as well as those inherent therein. The present disclosure is representative of embodiments, which are exemplary, and are not intended as limitations on the scope of the present disclosure. Changes and other uses will occur to those skilled in the art which are encompassed within the spirit of the present disclosure as defined by the scope of the claims.

No admission is made that any reference, including any non-patent or patent document cited in this specification, constitutes prior art. It will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method of selectively heating a lesion or tumor using laser therapy treatment, the method comprising:
administering plasmonic metal nanoplatforms to a subject having the lesion or tumor, whereby said plasmonic metal nanoplatforms selectively accumulate in the lesion or tumor, and
after selective accumulation of the plasmonic metal nanoplatforms in the lesion or tumor, administering laser therapy treatment to the lesion or tumor.

2. The method of claim 1, wherein the plasmonic metal nanoplatforms are selected from the group consisting of metal nanostars, metal nanorods, metal nanocaps, metal nanoshells, nanospheres, nanocages, nanotriangles, nanoplates.

3. The method of claim 1, wherein the metal of the plasmonic metal nanoplatforms comprises gold, silver, copper or a combination thereof.

4. The method of claim 1, wherein the plasmonic metal nanoplatforms comprise one or more of a paramagnetic nucleus and a bioreceptor.

5. The method of claim 1, wherein the plasmonic metal nanoplatforms have a tunable plasmonic absorption band in the near infrared region at or near 1000 nm.

6-8. (canceled)

9. The method of claim 1, wherein the plasmonic metal nanoplatforms are administered via infusion.

10. The method of claim 1, wherein the laser therapy treatment comprises Laser Interstitial Thermal Therapy (LITT).

11. The method of claim 1, wherein the lesion or tumor is an intracranial tumor or an intracranial lesion that is not a tumor, wherein the intracranial lesion is one or more of epileptic foci, tubers, cavernous malformations, arteriovenous malformations, and abscesses.

12. (canceled)

13. (canceled)

14. Method of enhancing absorption of excitation light from laser therapy in a lesion or tumor being treated with said laser therapy, the method comprising:
administering plasmonic metal nanoplatforms to a subject having the lesion or tumor to be treated with laser therapy, whereby said plasmonic metal nanoplatforms selectively accumulate in the lesion or tumor having different and/or irregular shapes by filling the contours of the lesion or tumor, and
treating the lesion or tumor in a conformal way with laser therapy whereby the excitation light from the laser therapy is strongly absorbed by the plasmonic metal nanoplatforms accumulated in the lesion or tumor thereby filling contours of the lesion or tumor.

15. The method of claim 14, wherein the metal of the plasmonic metal nanoplatforms comprises gold, silver, copper or a combination thereof.

16. The method of claim 14, wherein the plasmonic metal nanoplatforms comprise one or more of a bioreceptor or a paramagnetic nucleus.

17. (canceled)

18. The method of claim 14, wherein the plasmonic metal nanoplatforms are administered via infusion.

19. The method of claim 14, wherein the laser therapy treatment comprises Laser Interstitial Thermal Therapy (LITT).

20. The method of claim 14, wherein the lesion or tumor is an intracranial tumor or an intracranial lesion that is not a tumor, wherein the intracranial lesion is one or more of epileptic foci, tubers, cavernous malformations, arteriovenous malformations, and abscesses.

21-25. (canceled)

26. Method of enhancing absorption of excitation light from laser therapy in a lesion or tumor being treated with said laser therapy, the method comprising:
administering plasmonic metal nanoplatforms to a subject having the lesion or tumor to be treated with laser therapy, whereby said plasmonic metal nanoplatforms selectively accumulate in the lesion or tumor, and
treating the lesion or tumor with laser therapy whereby the excitation light from the laser therapy is absorbed by the plasmonic metal nanoplatforms accumulated in the lesion or tumor thereby inducing photon-to-heat conversion in the plasmonic metal nanoplatforms, thus transforming them into heat sources, leading to further heat propagation in tissue, lesion or tumor thereby inducing a larger treatment area.

27. The method of claim 26, wherein the metal of the plasmonic metal nanoplatforms comprises gold, silver, copper, or a combination thereof.

28. The method of claim 26, wherein the plasmonic metal nanoplatforms comprise one or more of a bioreceptor and a paramagnetic nucleus.

29. The method of claim 26, wherein the plasmonic metal nanoplatforms are administered via infusion.

30. (canceled)

31. The method of claim 26, wherein the laser therapy treatment comprises Laser Interstitial Thermal Therapy (LITT).

32. The method of claim 26, wherein the lesion or tumor is an intracranial tumor or an intracranial lesion that is not a tumor, wherein the intracranial lesion is one or more of epileptic foci, tubers, cavernous malformations, arteriovenous malformations, and abscesses.

33. (canceled)
